(19) United States
(12) Patent Application Publication
(10) Pub. No.: US 2024/0335364 A1
(43) Pub. Date: Oct. 10, 2024

(54) COMPOSITIONS OF DICAFFEOYLQUINIC ACIDS WITH TOCOPHEROL

(71) Applicant: SYMRISE AG, Holzminden (DE)

(72) Inventors: Martina HERRMANN, Hameln (DE); Sabine LANGE, Holzminden (DE); Sandra GAEBLER, Höxter (DE); Sebastian BRUNCKE, Höxter (DE); Katharina STRIEWE, Holzminden (DE)

(73) Assignee: SYMRISE AG, Holzminden (DE)

(21) Appl. No.: 18/290,919

(22) PCT Filed: Jul. 23, 2021
(86) PCT No.: PCT/EP2021/070712

§ 371 Date: Jan. 22, 2024

(51) Int. Cl. A61K 8/37; A61K 8/67; A61Q 17/00; A61Q 19/08

(52) U.S. Cl. CPC A61K 8/375; A61K 8/678; A61Q 17/00; A61Q 19/08

(57) ABSTRACT

The present invention relates to a mixture comprising one or more dicaffeoylquinic acids and tocopherol. Further, the invention relates to a cosmetic composition, a composition for food or pleasure or a pharmaceutical composition comprising such a mixture. Moreover, the invention relates to the uses of dicaffeoylquinic acids, of mixtures comprising these or of compositions comprising such mixtures. Even further, the invention relates to the use of tocopherol for increasing the stability of one or more dicaffeoylquinic acids in a mixture or a composition.

The present invention relates to a mixture comprising one or more dicaffeoylquinic acids and tocopherol. Further, the invention relates to a cosmetic composition, a composition for food or pleasure or a pharmaceutical composition comprising such a mixture. Moreover, the invention relates to the uses of dicaffeoylquinic acids, of mixtures comprising these or of compositions comprising such mixtures. Even further, the invention relates to the use of tocopherol for increasing the stability of one or more dicaffeoylquinic acids in a mixture or a composition.

Cells such as keratinocytes and fibroblasts constantly produce many different proteins. Chaperones and other cellular tools help the proteins to take their correct functional shape.

Heat shock proteins (HSPs) are proteins that function as molecular chaperones and participate in the folding of newly synthesized proteins and proteins with partially damaged structure. They play also a role in preventing the aggregation of incorrectly folded or partially unfolded proteins, assembly and disintegration of supramolecular protein structures, transport of proteins through membranes, and also unfolding of damaged proteins for their subsequent catabolism

Aging is amongst others characterized by a decreased number of chaperones, which leads to increased errors in protein folding and fewer functional proteins resulting in reduced metabolic efficacy in the concerned tissue.

Organisms and cells also have to react to various stress conditions, such as environmental, metabolic and pathophysiological stress, by selectively upregulating HSPs. HSPs were initially identified by their increased expression after a heat shock (usually one hour or more at temperatures of 3-5° C. above normal). The assumption that HSPs protect cells from heat damage is supported by the following facts: 1) HSP expression occurs in parallel with the development and decline of thermal tolerance (resistance to killing by heat); 2) mutation or inactivation of HSPs impairs a cell's ability to survive at high temperatures; 3) overexpression of HSPs can often improve a cell's ability to withstand high temperatures.

This heat shock response was then identified as a more general stress response, being triggered in different ways by a variety of stressful conditions affecting the cells.

Human skin is exposed to an environment that varies in humidity from 100 to 0%, leading to seasonal variations in the condition of the skin.

Humidity is the concentration of water vapour present in the air. It depends on the temperature and pressure. At 100% relative humidity, the air is saturated and is at its dew point. Relative humidity in the desert is between 10 and 30% and the humidity of cold winter outdoor air can be below 10%. The optimum humidity in living and working rooms e.g.

offices is between 40 and 60%. Low (<30%), very low (<20%) and extremely low (<10%) humidity can create discomfort, skin issues and health issues.

Air conditioning reduces the temperature and thereby discomfort, but at the same time, humidity is significantly reduced. Heating cold air indoors by e.g. central heating or other heating systems also decreases relative humidity levels to below 30% if no re-moisturing measures are taken.

Exposure to a low humidity environment creates an osmotic gradient across the stratum corneum, which is known to modulate cutaneous barrier function. One of the consequences of osmotic shock is loss of cellular water and denaturation of intracellular proteins. The pattern of osmotic stress-induced and heat-induced increase in HSP mRNA levels is quite similar.

Also cold shock (15° C. and below) was shown to stimulate a stress response in human epidermis altering the spectrum of proteins expressed and inducing the synthesis of HSPs.

These proteins are classified into six major families based on their molecular weight, i.e. HSP100, HSP90, HSP70, HSP60, HSP40 and small heat shock proteins (sHSPs). sHSPs have subunit molecular weights of 12-43 kDa.

An important function of many sHSPs is their ability to prevent aggregation of proteins and polypeptides. Inside the cell, they are constitutively expressed at a basal level but their expression is upregulated significantly during stress conditions such as elevated temperature. They are considered to be the cell's first line of defence against the deleterious effects of protein unfolding. Human sHSPs have very different characteristics with respect to their heat-induced expression, tissue and intracellular localization, structure, substrate preference and function. Because of this difference, human sHSPs have different capacities to protect against acute and different types of chronic (disease-related) stress.

HSPB2 (also known as HSP27) protects against stress-induced protein aggregation. HSP27 is found in intact skin mainly in the granular and prickle-cell layer of the epidermis. Melanocytes, dermal fibroblasts and endothelial cells do not express detectable amounts of HSP27. In hair follicles, staining is mainly confined to the outer root sheath and to the infundibular epithelium. The inducibility of HSP27 was shown to decrease with age by a comparison of biopsies from the upper inner arm of healthy young (age 19-28) and old (age 55-65) volunteers.

HSPB8 (also known as HSP22) is a relatively new member of the family of sHSPs. It is expressed in skin at lower levels, has chaperone activity, interacts with most sHSPs and contributes to the proteolytic degradation of unfolded proteins, involving proteosomes or autophagy regulation.

αB-crystallin (CRYAB, also known as HSPB5) is constitutively expressed in many tissues and has anti-apoptotic properties and chaperone activity. It can form oligomers with other HSPs, namely with HSP27.

HSP27 is also a regulator of biologic function beyond stress response. Its expression correlates with keratinocyte differentiation and increases continuously from the basal layer to the stratum granulosum. Keratinocyte differentiation leads to formation of the cornified layer and with this to the formation of a competent epidermal barrier, which is crucial to prevent entry of noxious stimuli.

The body surface of terrestrial animals and humans is exposed to air and to mechanical stress, both incompatible with the persistence of living cells at the direct interface between an organism and its environment. Furthermore, the typical air humidity is below the water concentration of the skin. Thus, the surrounding air constantly deprives the skin of its moisture, which is even stronger the lower the humidity. The stratifying epidermis of the skin physically separates the organism from its environment and serves as its first line of structural and functional defence against dehydration, chemical substances, physical insults and microorganisms. The living cell layers of the epidermis are crucial in the formation and maintenance of the barrier on two different levels. First, keratinocytes ultimately form the outermost protective dead layer of the skin through a complex spatial and temporal differentiation process. Impairment of this differentiation results in a reduced stratum corneum (SC) barrier function, as can be seen e.g. in atopic dermatitis. Second, the living cell layers themselves form a barrier by providing tight mechanical cohesion between the cells of the same and different epidermal layers. To establish this barrier, the viable cells have to connect to each other by intercellular junctions that link intercellular contacts to the cytoskeleton, such as tight junctions, (corneo) desmosomes and adherens junctions.

Tight Junctions (TJ) are occluding junctions. They seal the intercellular spaces between epithelial cells and the ‘tightness’ of this structure is dynamically regulated by environmental factors and addresses physiologic needs. A number of TJ proteins have been identified in human (and/or murine) epidermis and their cultured keratinocytes. They include claudins (CLDN), such as CLDN1, Occludin (OCLN) TJ associated MARVEL protein (TAMP) and junctional adhesion molecule (JAM) transmembrane families as well as several TJ plaque proteins (e.g. Zonula occludens/ZO and cingulin). Interestingly, most of the TJ proteins, identified by immunostaining of the epidermis, are localized to the cell-cell borders of the stratum granulosum (e.g., CLDN-1,-4,-6,-7,-11,-12, and-18, occludin, ZO-1, ZO-2, cingulin), where the functional TJ barrier has been found. Functional evidence that epidermal barrier function requires a tight junction component came from claudin-1 deficient mice, which die of massive transepidermal water loss (TEWL) due to impaired barrier function of the stratum granulosum. By using cultured keratinocytes, it was shown that TJs form a barrier to water and molecules of different size as well as ions.

Desmosomes are “mechanical” junctions, involved primarily in cell cohesion. They are composed of the desmosomal cadherins, which, similar to the classical cadherins of adherens junctions, are part of the cadherin superfamily. Desmogleins (DSG) 1-4 and desmocollins (DSC) 1-3 are found in the human epidermis. The intracellular ends of desmosomal cadherins are inserted in the molecular network of adaptor proteins forming desmosomal plaques, to which keratin filaments bind. As keratinocytes move through the epidermal layers, they constantly form and retrieve desmosomes at the cell periphery. During this turnover, the molecules that compose junctions (even without physical dissociation of the structure) are also constantly replaced. According to the level of keratinocyte differentiation, desmogleins 2 and 3 from the lower epidermal compartment are progressively substituted by desmogleins 1 and 4 in the upper viable epidermal layers. In the same way, desmocollin 3 is replaced by desmocollin 1. This differentiation-dependent composition of desmosomes coincides with the increase of their mechanical stability.

Adherens junctions are intercellular structures that couple intercellular adhesion to the cytoskeleton thereby creating a transcellular network that coordinate the behaviour of a population of cells. Adherens junctions are dynamic entities and function as signal platforms that regulate cytoskeletal dynamics and cell polarity. As such, they regulate a diverse range of other cellular processes next to adhesion, such as cell shape, division, growth, apoptosis and barrier function. The molecular basis of adherens junctions is formed by two cell adhesion receptor complexes, the classical cadherin/catenin complex and the nectin/afadin complex, which both can link to the actin cytoskeleton. Classical cadherins are single transmembrane Ca2+-dependent cell adhesion molecules that at their cytoplasmic face interact with catenins. Two types of classical cadherins are expressed in the epidermis: P-cadherin (cadherin 3), expressed in the basal layer mainly around and in hair follicles, and E-cadherin (cadherin 1) found in all layers of the epidermis.

Agents that reinforce epidermal integrity by stimulating junctional genes and proteins and thereby increase defence functions also promote tissue homoeostasis and therefore can be expected to be also beneficial for tissue functionality. Moreover, aging of the skin is also associated with a loss of skin moisture, i.e. a constant loss of water in the skin ultimately adds to skin aging.

As a chemical hindrance, the epidermis possesses acidic pH, highly organized lipids and various host defense peptides, also known as antimicrobial peptides. Human beta-defensins, one of the most important host defense peptide families found in the skin, are wellknown for their broad-spectrum microbicidal activities. They are cationic peptides mainly produced by epithelial cells of many organs including skin, lung, kidney, pancreas, uterus, eye, and nasal and oral mucosa. Human B-defensin 1 peptide (hBD-1) is encoded by the DEFB1 locus and acts against gram-positive and negative bacteria. Moreover, recent work identified the potential role of hBDs in the regulation and maintenance of the skin barrier function.

WO 2013/115683 A2 describes the induction of heat shock protein synthesis (focus high molecular weight HSPs=HSP60, HSP70, HSP90 and HSP100) in human and animal cells by at least one phenol compound selected out of the group of cinnamic acid derivatives, preferably cinnamic acid derivatives such as chlorogenic, ferulic or caffeic acid, curcumin or the phenylethyl ester of caffeic acid. The mixture of phenol compounds can also be represented by a plant extract, e.g. coffee bean extract, preferably green coffee bean extract.

WO 2018/196993 A1 describes dried yarrow (Achillea millefolium) fresh plant press juice as upregulator of small heat shock proteins (HSPs) such as HSP27 and antimicrobial proteins and peptides (AMPs) such as for example beta-defensin 1 and S100 calcium binding protein A8 at gene level.

Dried yarrow fresh plant press juice is characterised by the presence of a relatively high content of polyphenols (16.4%), especially phenolcarboxylic acids including the group of monocaffeoylquinic acids and the group of dicaffeoylquinic acids. It further contains rutin, proteins, amino acids and minerals.

Caffeoylquinic acids are a broad class of very common secondary metabolites that have been found in edible and medicinal plants from various families. Molecules consisting of one caffeic acid molecules linked to one quinic acid molecule are called monocaffeoylquinic acids. Dicaffeoylquinic acids (molecular formula C25H24012) are characterized by two caffeic acid molecules linked via ester bonds to one quinic acid molecule.

Caffeoylquinic acids are present e.g. in apples, stone fruits (peaches, nectarines, plums, lychees, mangoes, cherries, Cydonia oblonga), berry fruits (blueberries, blackcurrants, blackberries, bilberries), coffee beans, mate (IIex paraguariensis), Lonicera japonica, brassica vegetables (kale, cabbage and Brussels sprouts), apiaceae (celery, carrots, caraway and coriander) and other miscellaneous vegetables like corn salad, anise stars and potato. Nevertheless, despite their high prevalence, they are believed to be the most characteristic constituents of plants from the Asteraceae family, such as artichoke (Cynara scolymus L.), Cynara cardunculus, black salsify (Scorzonera hispanica L.), common yarrow (Achillea millefolium L.), milk thistle (Silybum marianum L. Gaertner), coltsfoot (Tussilago farfara L.), tansy (Tanacetum vulgare L.) and chamomile (Matricaria chamomilla L.). Among all caffeoylquinic acids, the monocaffeoylquinic acid chlorogenic acid is the most ubiquitous.

Accumulated evidence demonstrate that these compounds have a wide range of biological activities, such as antioxidant, antibacterial, antiparasitic, neuroprotective, anti-inflammatory, anticancer, antiviral, and antidiabetic effects. However, one of the significant and challenging features of caffeoylquinic acids is their chemical instability.

Although there are already known several agents which induce heat shock proteins, there is a constant demand for new, highly effective already at low use levels, safe and stable

HSP inducers, especially sHSP inducers. These agents will support proper protein and thereby tissue functionality (protein homeostasis) in general, upon aging and also in stress situations.

Agents that in parallel also support proper barrier function e.g. cutaneous barrier function will be even more requested by the industry.

Another drawback of many agents, which induce heat shock proteins, is that these agents are not very stable, particularly in compositions, in which these agents are provided. As an example, several such agents are not stable in typical cosmetic formulations. Thus, to make advantageous use of the HSP-inducing effect of these agents, they need to be provided in a stabilized form in such compositions.

The primary object of the present invention was thus to provide novel mixtures comprising agents, which induce HSPs and/or support a proper/improve the barrier function, and simultaneously, in which these agents are present in a stabilized form.

The primary object of the present invention is achieved by a mixture comprising or consisting of
(a) one or more dicaffeoylquinic acid(s),
   preferably wherein one, two, three or more or all of the dicaffeoylquinic acid(s) is/are selected from the group consisting of 3,4-dicaffeoylquinic acid, 3,5-dicaffeoylquinic acid, 1,5-dicaffeoylquinic acid and 4,5-dicaffeoylquinic acid,
   further preferably wherein one, two or three or all of the dicaffeoylquinic acid(s) is/are selected from the group consisting of 3,4-dicaffeoylquinic acid, 3,5-dicaffeoylquinic acid and 4,5-dicaffeoylquinic acid,
   particularly preferably wherein one or two or all of the dicaffeoylquinic acid(s) is/are selected from the group consisting of 3,4-dicaffeoylquinic acid and 3,5dicaffeoylquinic acid, and
(b) tocopherol,
   wherein the weight ratio of the total weight of component (a) to the total weight of component (b) is in a range of from 1:1000 to 5:1, preferably in a range of from 1:800 to 2:1, particularly preferably in a range of from 1:600 to 1:1, especially preferably in a range of from 1:500 to 1:2, more preferably in a range of from 1:300 to 1:5, further preferably in a range of from 1:250 to 1:10; especially preferably in a range of from 1:100 to 1:20 even more preferably in a range of from 1:75 to 1:25. Furthermore in a mixture according to the invention, the ratio described above may be any combination of any upper and any lower limit or any upper and any other upper limit or any lower and any other lower limit, as described above.

Preferably, components (a) and (b) are present in the mixture in a ratio, in which the components act synergistically, as described herein.

The compound 3,4-dicaffeoylquinic acid is further described by CAS number 57378-72-0 and preferably includes all possible stereoisomers thereof.

The compound 3,5-dicaffeoylquinic acid is further described by CAS number 2450-53-5 and preferably includes all possible stereoisomers thereof.

The compound 1,5-dicaffeoylquinic acid is further described by CAS number 19870-46-3 and preferably includes all possible stereoisomers thereof.

The compound 4,5-dicaffeoylquinic acid is further described by CAS number 14534-61-3 and preferably includes all possible stereoisomers thereof.

The term “tocopherol” describes a class of organic chemical compounds (more precisely, various methylated phenols), many of which have vitamin E activity. Thus, the term “tocopherol” includes several compounds such as tocopherols (alpha, beta, gamma, and delta-tocophenol) and tocotrienols (alpha, beta, gamma, and delta-tocotrienol). However, the term “tocopherol” does not include tocopheryl acetate.

It was surprisingly found that dicaffeoylquinic acids are highly potent agents for inducing the expression of HSPs as shown in Example 1, whereas also surprisingly, monocaffeoylquinic acids were not able to induce HSPs as shown in Example 2.

Likewise, it was surprisingly found that dicaffeoylquinic acids, but not monocaffeoylquinic acids, also induce “mechanical”, desmosomal and tight junctional proteins, as shown by induction of DSG1, DSC1, CLDN1 and OCDN, as shown in Examples 1 and 2 which are involved in an improved barrier function and thereby reduction of stress induced protein damages.

Even more surprisingly, it was found that dicaffeoylquinic acids in combination with tocopherol lead to a synergistic upregulation of HSPs, desmosomal and tight junctional protein levels, as shown in Examples 4 to 8. This is especially noticeable, as tocopherol alone does not have such an effect.

It is preferred that a mixture according to the invention comprises 0.1 to 85 wt.-%, preferably 0.2 to 70 wt.-%, particularly preferably 0.25 to 50 wt.-%, further preferably 0.5 to 40 wt.-%, more preferably 1 to 20 wt.-%, even further preferably 2 to 10 wt.-%, especially preferably 2.5 to 5 wt.-% of component (a), related to the total weight of the mixture and/or that a mixture according to the invention comprises 15 to 99.9 wt.-%, preferably 20 to 99 wt.-%, particularly preferably 25 to 95 wt.-%, further preferably 30 to 90 wt.-%, more preferably 40 to 80 wt.-%, even further preferably 50 to 75 wt.-%, especially preferably 55 to 65 wt.-% of component (b), related to the total weight of the mixture.

Additionally or alternatively, it is preferred that component (a) and component (b) are present in the mixture in amounts, in which component (a) and component (b) act synergistically, as described herein.

Preferably, the mixture according to the invention consists of component (a) and component (b).

Components (a) and (b) may be of synthetic or of natural origin. In case, compound (a) and/or (b) is of natural origin, it may be present in a plant extract. In this case, the plant extract can be added to provide a mixture according to the invention. Further, compound (a) and/or (b) may be present in more than one plant extract. In this case, the plant extracts (i.e. a mixture of plant extracts) can all be added to provide a mixture according to the invention. As shown in Example 3, the effect described herein is also obtained when components (a) and/or (b) are present as a plant extract.

Preferably, components (a) and (b) are present in the same or in a different extract, or in the same or in a different mixture of extracts.

Preferably, such a plant extract is obtained from a plant selected from the group consisting of yarrow, arnica, coffee, elderflower, ivy, honeysuckle, chrysanthemum, artichoke or mixtures thereof. Preferably, a plant extract from coffee is a coffee bean extract, particularly preferably a green coffee bean extract. Preferably, a plant extract from ivy is an extract from ivy leaf. Preferably, a plant extract from honeysuckle is a plant extract from honeysuckle flower.

Therefore, it is preferred that the mixture according to the invention comprises a plant extract, preferably a plant extract obtained from yarrow, arnica, coffee, elderflower, ivy, honeysuckle, chrysanthemum, artichoke or mixtures of such plant extracts and wherein the plant extract or mixture of plant extracts comprises component (a), and/or that the mixture according to the invention comprises a plant extract or mixture of plant extracts and wherein the plant extract or mixture of plant extracts comprises component (b). Preferably in this case, component (a) and/or (b) of the mixture according to the invention is only present in the plant extract(s), i.e. no additional component (a) and/or (b) is present in the mixture according to the invention, except for component (a) and/or (b) of the plant extract(s).

Additionally it is preferred that the a plant extract as described herein comprises at least 50 wt.-%, preferably at least 75 wt.-%, particularly preferably at least 90 wt.-%, further preferably at least 95 wt.-%, more preferably at least 97 wt.-%, even further preferably at least 98 wt.-%, especially preferably at least 99 wt.-% of plant based components, based on the total weight of the extract.

Also preferred is that the a plant extract as described herein comprises at most 99.5 wt.-%, preferably at most 99 wt.-%, further preferably at most 98 wt.-%, particularly preferably at most 97 wt.-%, more preferably at most 95 wt.-%, further preferably at most 90 wt.-%, even further preferably at most 75 wt.-%, especially preferably at most 50 wt.-%, particularly preferably at most 30 wt.-%, further preferably at most 25 wt.-%, particularly preferably at most 20 wt.-%, more preferably at most 10 wt.-%, further preferably at most 5 wt.-%, even further preferably at most 1 wt.-% of plant based components, based on the total weight of the extract, wherein the term “at most” excludes the possibility that no plant based component is present.

The term “plant based components” describes components, which are present in the plant material, from which the extract was obtained, but excludes dicaffeoylquinic acids and tocopherol. Preferably, the term “plant based components” describes components selected from the group consisting of chlorophyll, rubisco, cellulose, sugars such as mono-, di-, tri or oligosaccharides, sugar alcohols, polysaccharides, amino acids, peptides, proteins, minerals, phenolics, tannins, flavonoids, isoflavonoids, organic acids, fatty acids, terpenes such as monoterpenes (C10), sesquiterpenes (C15), diterpenes (C20) or triterpenes (C30), carotenoids, alkaloids, amines.

Thus, the mixture according to the invention may preferably comprise one or more plant extract(s), comprising component (a), and synthetically obtained component (b). Additionally or alternatively, the mixture according to the invention may preferably comprise one or more plant extract(s), comprising component (b), and synthetically obtained component (a). Additionally or alternatively, the mixture according to the invention may preferably comprise one or more plant extract(s), comprising component (a), and one or more plant extract(s), comprising component (b). Additionally or alternatively, the mixture according to the invention may preferably comprise synthetically obtained component (a) and synthetically obtained component (b).

Further preferably, component (b) may be naturally obtained, however, not be present in an extract, as it is described above. Thus, the mixture according to the invention may preferably comprise synthetically obtained component (a) and naturally obtained component (b). Additionally or alternatively, the mixture according to the invention may preferably comprise one or more plant extract(s), comprising component (a), and naturally obtained component (b).

Furthermore, the invention relates to a composition, preferably a cosmetic composition, a composition for food or pleasure or a pharmaceutical composition comprising a mixture according to the invention.

It is preferred that a composition according to the invention comprises 0.000001 to 75 wt.-%, preferably 0.00005 to 50 wt.-%, particularly preferably 0.00001 to 25 wt.-%, especially preferably 0.0005 to 10 wt.-%, further preferably 0.0001 to 1 wt.-%, even further preferably 0.0005 to 0.5 wt.-% of component (a), related to the total weight of the composition and/or that a composition according to the invention comprises 0.0001 to 99 wt.-%, preferably 0.001 to 95 wt.-% particularly preferably 0.01 to 90 wt.-%, especially preferably 0.01 to 50 wt.-%, further preferably 0.1 to 25 wt.-%, more preferably 0.1 to 10 wt.-%, further preferably 0.1 to 5 wt.-%, even further preferably 0.1 to 1 wt.-% of component (b), related to the total weight of the composition.

Components (a) and (b) may be of synthetic or of natural origin. In case, compound (a) and/or (b) is of natural origin, it may be present in a plant extract. In this case, the plant extract can be added to provide a composition according to the invention. Further, compound (a) and/or (b) may be present in more than one plant extract. In this case, the plant extracts (i.e. a mixture of plant extracts) can all be added to provide a composition according to the invention. As shown in Example 3, the effect described herein is also obtained when components (a) and/or (b) are present as a plant extract.

Preferably, components (a) and (b) are present in the same or in a different extract, or in the same or in a different mixture of extracts.

Preferably, such a plant extract is obtained from a plant selected from the group consisting of yarrow, arnica, coffee, elderflower, ivy, honeysuckle, chrysanthemum, artichoke or mixtures thereof. Preferably, a plant extract from coffee is a coffee bean extract, particularly preferably a green coffee bean extract. Preferably, a plant extract from ivy is an extract from ivy leaf. Preferably, a plant extract from honeysuckle is a plant extract from honeysuckle flower.

Therefore, it is preferred that the composition according to the invention comprises a plant extract, preferably a plant extract obtained from yarrow, arnica, coffee, elderflower, ivy, honeysuckle, chrysanthemum, artichoke or mixtures of such plant extracts and wherein the plant extract or mixture of plant extracts comprises component (a), and/or that the composition according to the invention comprises a plant extract or mixture of plant extracts and wherein the plant extract or mixture of plant extracts comprises component (b). Preferably in this case, component (a) and/or (b) of the composition according to the invention is only present in the plant extract(s), i.e. no additional component (a) and/or (b) is present in the composition according to the invention, except for component (a) and/or (b) of the plant extract(s).

Additionally it is preferred that the a plant extract as described herein comprises at least 50 wt.-%, preferably at least 75 wt.-%, particularly preferably at least 90 wt.-%, further preferably at least 95 wt.-%, more preferably at least 97 wt.-%, even further preferably at least 98 wt.-%, especially preferably at least 99 wt.-% of plant based components, based on the total weight of the extract.

Also preferred is that the a plant extract as described herein comprises at most 99.5 wt.-%, preferably at most 99 wt.-%, further preferably at most 98 wt.-%, particularly preferably at most 97 wt.-%, more preferably at most 95 wt.-%, further preferably at most 90 wt.-%, even further preferably at most 75 wt.-%, especially preferably at most 50 wt.-%, particularly preferably at most 30 wt.-%, further preferably at most 25 wt.-%, particularly preferably at most 20 wt.-%, more preferably at most 10 wt.-%, further preferably at most 5 wt.-%, even further preferably at most 1 wt.-% of plant based components, based on the total weight of the extract, wherein the term “at most” excludes the possibility that no plant based component is present.

The term “plant based components” describes components, which are present in the plant material, from which the extract was obtained, but excludes dicaffeoylquinic acids and tocopherol. Preferably, the term “plant based components” describes components selected from the group consisting of chlorophyll, rubisco, cellulose, sugars such as mono-, di-, tri or oligosaccharides, sugar alcohols, polysaccharides, amino acids, peptides, proteins, minerals, phenolics, tannins, flavonoids, isoflavonoids, organic acids, fatty acids, terpenes such as monoterpenes (C10), sesquiterpenes (C15), diterpenes (C20) or triterpenes (C30), carotenoids, alkaloids, amines.

Thus, the composition according to the invention may comprise one or more plant extract(s) comprising component (a) and synthetically obtained component (b). Additionally or alternatively, the composition according to the invention may comprise one or more plant extract(s) comprising component (b) and synthetically obtained component (a). Additionally or alternatively, the composition according to the invention may comprise one or more plant extract(s) comprising component (a) and one or more plant extract(s) comprising component (b). Additionally or alternatively, the composition according to the invention may comprise synthetically obtained component (a) and synthetically obtained component (b).

A cosmetic composition according to the invention may preferably be understood as a composition, which is suitable for topical application to the skin of a human being or an animal, in particular to achieve a cosmetic effect. A cosmetic composition according to the invention may comprise one or more cosmetically acceptable carrier.

Preferably such cosmetically acceptable carriers are carriers other than water, more preferably carriers selected from the group consisting of glycols, aliphatic esters, preferably polyethyleneglycol esters and polyethyleneglycol ethers or mixtures thereof, in particular cosmetically acceptable carriers for enhancing the bioavailability of one, more or all of the further components of the cosmetic composition.

Particularly preferably, cosmetically acceptable carriers are selected from the group consisting of
diols, preferably alkane diol(s), particularly preferably such having 3 to 10 carbon atoms, preferably selected from the group consisting of 1,2-propylene glycol, 2-methylpropane-1,3-diol, 1,2-butylene glycol, 1,3-butanediol, 1,2-pentanediol, 1,3-pentanediol, 1,5-pentanediol, 2,4-pentanediol, 2-methyl-pentane-2,4-diol, 1,2-hexanediol, 1,6-hexanediol, 1,2-octanediol, 1,2-heptanediol, 1,2-decanediol;
aliphatic esters, preferably aliphatic esters having 6 to 36 carbon atoms, preferably monoesters, diesters or triesters, preferably selected from the group consisting of diethyl phthalate diethylhexyl 2,6-naphthalate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 3,5,5-trimethylhexyl 3,5,5-trimethylhexanoate, 2-ethylhexyl isononanoate, 2-ethylhexyl 3,5,5-trimethylhexanoate, 2-ethylhexyl 2-ethylhexanoate, 2-ethylhexyl palmitate, 2-ethylhexyl laurate, 2-hexyldecyl stearate, cetearyl ethylhexanoate, stearyl isononanoate, palmityl isononanoate, cetearyl isononanoate, palmityl 3,5,5-trimethylhexanoate, stearyl 3,5,5-trimethylhexanoate, cetearyl 3,5,5-trimethylhexanoate, stearyl heptanoate, stearyl caprylate, 2-octyldodecyl palmitate, oleyl oleate, oleyl erucate, erucyl oleate, erucyl erucate, 2-ethylhexyl isostearate, isotridecyl isononanoate, 2-ethylhexyl cocoate, C12-15-alkyl benzoates, cetyl palmitate, triethyl citrate, triacetin (triacetyl citrate), benzyl benzoate, benzyl acetate, vegetable oils (preferably olive oil, sunflower oil, soya oil, groundnut oil, rapeseed oil, almond oil, palm oil, coconut oil, palm kernel oil) and triglycerides, in particular glyceryl stearate, glyceryl triisononanoate, glyceryl laurate or triglycerides with identical or different C₆ to C₁₀ fatty acid radicals (so-called medium-chain triglycerides, in particular caprylic/capric triglyceride, like glyceryl tricaprylate, glyceryl tricaprate);
branched and unbranched alkyl or alkenyl alkohols, preferably selected from the group consisting of decanol, decenol, octanol, octenol, dodecanol, dodecenol, octadienol, decadienol, dodecadienol, oleyl alcohol, ricinoleyl alcohol, erucyl alcohol, stearyl alcohol, isostearyl alcohol, cetyl alcohol, lauryl alcohol, myristyl alcohol, arachidyl alcohol, linoleyl alcohol, linolenyl alcohol, hexyldecanol, octyldodecanol (in particular 2-octyl-1-dodecanol) and cetearyl alcohol and behenyl alcohol;
branched and unbranched hydrocarbons and waxes, cyclic or linear silicone oils and dialkyl ethers, particularly such having 6 to 24 carbon atoms, preferably selected from the group consisting of jojoba oil, isoeicosane, dicaprylyl ether, mineral oil, petrolatum, squalane, squalene, cyclomethicone, decamethylcyclopentasiloxane, undecamethylcyclotrisiloxane, polydimethylsiloxane and poly (methyl-phenyl) siloxane; and
miscellaneous other solvents like acetone, methylpropyl ketone, dipropyl ketone, dimethyl sulfoxide, glycerine carbonate, propylene carbonate, butylene carbonate glycerine formal, solketal, 2-ethyl hexanol, 2-butyl octanol, 2-hexyl decanol or 2-octyl dodecanol.

Preferably, compositions comprising one or more cosmetically acceptable carriers as described above are easy to handle and stable over a long period of time.

Preferably, a cosmetic composition according to the invention comprises suitable auxiliary substances and additives, such as, for example preservatives, in particular those described in US 2006/0089413, antimicrobial agents, such as e.g. antibacterial agents or agents to treat yeast and mold, in particular those described in WO 2005/123101, antiacne and sebum reducing agents, in particular those described in WO 2008/046791, compounds against ageing of the skin, in particular those described in WO 2005/123101, antidandruff agents, in particular those described in WO 2008/046795, antiirritants (antiinflammatory agents, irritation-preventing agents, irritation-inhibiting agents), in particular those described in WO 2007/042472 and US 2006/0089413, antioxidants, in particular those described in WO 2005/123101, carrier materials, in particular those described in WO 2005/123101, chelating agents, in particular those described in WO 2005/123101, deodorizing agents and antiperspirants, in particular those described in WO 2005/123101, moisture regulators (moisture-donating agents, moisturizing substance, moisture-retaining substances), in particular those described in WO 2005/123101, osmolytes, in particular those described in WO 2005/123101, compatible solutes, in particular those described in WO 01/76572 and WO 02/15868, proteins and protein hydrolysates, in particular those described in WO 2005/123101 and WO 2008/46676, skin-lightening agents, in particular those described in WO 2007/110415, skin-tanning agents, in particular those described in WO 2006/045760, cooling agents, in particular those described in WO 2005/123101, skincooling agents, in particular those described in WO 2005/123101, skin warming agents, in particular those described in WO 2005/123101, UV-absorbing agents, in particular those described in WO 2005/123101, UV filters, in particular those described in WO 2005/123101, benzylidene-beta-dicarbonyl compounds in accordance with WO 2005/107692 and alpha-benzoyl-cinnamic acid nitriles in accordance with WO 2006/015954, insect repellents, in particular those described in WO 2005/123101, plant parts, plant extracts, in particular those described in WO 2005/123101, vitamins, in particular those described in WO 2005/123101, emulsifiers, in particular those described in WO 2005/123101, gelling agents, in particular those described in WO 2005/123101, oils in particular those described in WO 2005/123101, waxes in particular those described in WO 2005/123101, fats in particular those described in WO 2005/123101, phospholipids, in particular those described in WO 2005/123101, saturated fatty acids and mono-or polyunsaturated fatty acids and a-hydroxy acids and polyhydroxy-fatty acids and esters of saturated and/or unsaturated branched and/or unbranched alkane carboxylic acids, in particular those described in WO 2005/123101, surface-active substances (surfactants) in particular those described in WO 2005/123101, skin repair agents comprising cholesterol and/or fatty acids and/or ceramides and/or pseudoceramides, in particular those described in WO 2006/053912, dyestuffs and colorants and pigments, in particular those described in WO 2005/123101, aroma chemicals and flavors and fragrances, in particular those described in S. Arctander, 20 Perfume and Flavor Chemicals, private publishing house, Montclair, N.J., 1969 and Surburg, Panten, Common Fragrance and Flavor Materials, 5th Edition, Wiley-VCH, Weinheim 2006, preferably those explicitly mentioned in US 2008/0070825, alcohols and polyols, in particular those described in WO 2005/123101, organic solvents, in particular those described in WO 2005/123101, silicones and silicone oils and silicone derivatives in particular those described in WO 2008/046676, virucides, abrasives, anticellulite agents, astringents, antiseptic agents, antistatics, binders, buffers, cell stimulants, cleansing agents, care agents, depilatory agents, softeners, enzymes, essential oils, in particular those described in US 2008/0070825, fibres, film-forming agents, fixatives, foamforming agents, foam stabilizers, substances for preventing foaming, foam boosters, gelforming agents, hair growth activators, hair growth inhibitors, hair care agents, hair-setting agents, hair-straightening agents, hair-smoothening, bleaching agents, strengthening agents, stain-removing agents, optically brightening agents, impregnating agents, dirt-repellent agents, friction-reducing agents, lubricants, opacifying agents, plasticizing agents, covering agents, polish, gloss agents, polymers in particular those described in WO 2008/046676, powders, peptides, mono-, di- and oligosaccharides, re-oiling agents, abrading agents, skin-soothing agents, skin-cleansing agents, skin care agents, skin-healing agents, skin-protecting agents, skin-softening agents, skin-smoothing agents, nourishing agents, skin-warming agents, stabilizers, detergents, fabric conditioning agents, suspending agents, thickeners, yeast extracts, algae or microalgae extracts, animal extracts, liquefiers, color-protecting agents, and electrolytes.

The cosmetic or pharmaceutical composition according to the invention can comprise cosmetic auxiliary substances and additives such as are conventionally used in such formulations, e.g. sunscreen agents, preservatives, bactericides, fungicides, virucides, cooling active compounds, insect repellents (e.g. DEET, IR 3225), plant extracts, plant parts, antiinflammatory active compounds, substances which accelerate wound healing (e.g. chitin or chitosan and derivatives thereof), film-forming substances (e.g. polyvinylpyrrolidones or chitosan or derivatives thereof), antioxidants, vitamins, 2-hydroxycarboxylic acids (e.g. citric acid, malic acid, L-, D-or dl-lactic acid), skin-colouring agents (e.g. walnut extracts or dihydroxyacetone), active compounds for promoting hair growth or inhibiting hair growth, skin care compositions (e.g. cholesterol, ceramides, pseuodceramides), softening, moisturizing and/or humectant substances, fats, oils, saturated fatty acids, mono-or polyunsaturated fatty acids, a-hydroxy acids, polyhydroxyfatty acids or derivatives thereof, waxes or other conventional constituents of a cosmetic or dermatological formulation, such as alcohols, polyols, polymers, foam stabilizers, electrolytes, organic solvents, silicone derivatives of chelating agents (e.g. ethylenediaminetetraacetic acid and derivatives), antidandruff active compounds (e.g. climbazole, ketoconazole, piroctonoleamine, zinc pyrithione), hair care agents, perfumes, substances for preventing foaming, dyestuffs, pigments which have a colouring action, thickening agents (advantageously silicon dioxide, aluminium silicates, such as e.g. bentonites, polysaccharides or derivatives thereof, e.g. hyaluronic acid, guar bean flour, xanthan gum, hydroxypropylmethylcellulose or allulose derivatives, particularly advantageously polyacrylates, such as e.g. Carbopols or polyurethanes), surface-active substances and emulsifiers.

Additionally or alternatively, a cosmetic composition according to the invention may comprise one or more additional substances, preferably selected from the group consisting of
anti-itch compounds;
steroidal anti-inflammatory substances of the corticosteroid type, in particular hydrocortisone, hydrocortisone derivatives such as hydrocortisone 17-butyrate, dexamethasone, dexamethasone phosphate, methylprednisolone or cortisone;
non-steroidal anti-inflammatory substances, in particular oxicams such as piroxicam or tenoxicam, salicylates such as aspirin, disalcid, solprin or fendosal, acetic acid derivatives such as diclofenac, fenclofenac, indomethacin, sulindac, tolmetin or clindanac, fenamates such as mefenamic, meclofenamic, flufenamic or niflumic, propionic acid derivatives such as ibuprofen, naproxen or benoxaprofen, pyrazoles such as phenylbutazone, oxyphenylbutazone, febrazone or azapropazone;
natural or naturally occurring anti-inflammatory substances or substances that alleviate reddening and/or itching, in particular extracts or fractions from camomile, Aloe vera, Commiphora species, Rubia species, willow, willowherb, oats, calendula, arnica, St John's wort, honeysuckle, rosemary, Passiflora incarnata, witch hazel, ginger or Echinacea, or single active compounds thereof;
alpha-bisabolol, apigenin, apigenin-7-glucoside, gingerols, shogaols, gingerdiols, dehydrogingerdiones, paradols, preferably [6]-paradol, natural avenanthramides, non-natural avenanthramides, preferably dihydroavenanthramide D, boswellic acid, phytosterols, glycyrrhizin, glabridin, cannabidiol (CBD) and licochalcone A, preferably in the form of pure substances,
skin care agents, preferably skin moisture retention regulators or skin repair agents, preferably selected from the group consisting of sodium lactate, urea and derivatives, glycerol, propylene glycol, 1,2-pentanediol, 1,2-hexanediol, 1,2-heptanediol and 1,2-octanediol, sugar alcohols, preferably C3-C6 sugar alcohols, collagen, elastin or hyaluronic acid, diacyl adipates, petrolatum, urocanic acid, lecithin, allantoin, panthenol, phytantriol, lycopene, (pseudo-) ceramides (preferably Ceramide 2, hydroxypropyl bispalmitamide MEA, cetyloxypropyl glyceryl methoxypropyl myristamide, N(1-hexadecanoyl)-4-hydroxy-L-proline (1-hexadecyl) ester, hydroxyethyl palmityl oxyhydroxypropyl palmitamide), glycosphingolipids, cholesterol, phytosterols, chitosan, chondroitin sulfate, lanolin, lanolin esters, amino acids, vitamin E and derivatives (preferably tocopherol, tocopheryl acetate), alpha-hydroxy acids (preferably citric acid, lactic acid, malic acid) and derivatives thereof, mono-, di- and oligosaccharides, preferably glucose, galactose, fructose, mannose, laevulose and lactose, polysugars, such as β-glucans, in particular 1,3-1,4-β-glucan from oats, alpha-hydroxyfatty acids triterpenic acids, such as betulic acid or ursolic acid, and algae extracts or single active compounds thereof;
physiological cooling agents, preferably selected from the group consisting of menthone glycerol acetal, menthyl lactate preferably I-menthyl lactate, in particular I-menthyl I-lactate), menthyl ethyl oxamate, substituted menthyl-3-carboxylic acid amides (e.g. menthyl-3-carboxylic acid N-ethylamide, Nα-(Lmenthanecarbonyl) glycine ethyl ester, 2-isopropyl-N-2,3-trimethylbutanamide, substituted cyclohexanecarboxylic acid amides, 3-menthoxypropane-1,2-diol, 2-hydroxyethyl menthyl carbonate, 2-hydroxypropyl menthyl carbonate, N-acetylglycine menthyl ester, isopulegol, menthyl hydroxycarboxylic acid esters (e.g. menthyl 3-hydroxybutyrate), monomenthyl succinate, monomenthyl glutarate, 2-mercaptocyclodecanone, menthyl 2-pyrrolidin-5-onecarboxylate, 2,3-dihydroxy-p-menthane, 3,3,5-trimethylcyclohexanone glycerol ketal, 3-menthyl 3,6-di- and-trioxaalkanoates, 3-menthyl methoxyacetate and icilin;
histamine receptor antagonists, serine protease inhibitors, TRPV1 antagonists, NK1 antagonists, cannabinoid receptor agonists and TRPV3 antagonists;
antioxidants, preferably selected from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. a-carotene, β-carotene, lycopene) and derivatives thereof, chlorogenic acid and derivatives thereof, liponic acid and derivatives thereof (e.g. dihydroliponic acid), aurothioglucose, propylthiouracil and other thiols (e.g. thioredoxin, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), (metal) chelators, e.g. α-hydroxy-fatty acids, palmitic acid, phytic acid, lactoferrin, a-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate, ascorbyl glycosides, such as e.g. 6-O-acyl-2-O-α-D-glucopyranosyl-L-ascorbic acid, 6-O-acyl-2-O-β-D-glucopyranosyl-Lascorbic acid, 2-O-α-D-glucopyranosylL-ascorbic acid or 2-O-β-D-glucopyranosyl-L-ascorbic acid), tocopherols and derivatives thereof (e.g. vitamin E acetate), vitamin A and derivatives thereof (vitamin A palmitate) as well as coniferylbenzoate of benzoin resin, rutic acid and derivatives thereof, a-glucosylrutin, quercetin and derivatives thereof, rosemary acid, carnosol, carnosol acid, resveratrol, caffeic acid and derivatives thereof, sinapic acid and derivatives thereof, ferulic acid and derivatives thereof, furfurylideneglucitol, butylhydroxytoluene, butylhydroxyanisole, nordihydroguaiac resin acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO4), selenium and derivatives thereof (e.g. selenium methionine), stilbenes and derivatives thereof (e.g. stilbene oxide, trans-stilbene oxide) and derivatives (salts, esters, ethers, sugars, nucleotides, nucleosides, peptides and lipids) of these active compounds mentioned or antioxidatively active extracts or fractions from plants, such as e.g. green tea, rooibos, honeybush, grape, rosemary, sage, Melissa, thyme, lavender, olive, oats, cocoa, ginkgo, ginseng, liquorice, honeysuckle, Sophora, Pueraria, Pinus, Citrus, Phyllanthus emblica or St. John's wort;
physiological warming (heating) agents, preferably selected from the group consisting of vanillyl alcohol n-butyl ether, vanillyl alcohol n-propyl ether, vanillyl alcohol isopropyl ether, vanillyl alcohol isobutyl ether, vanillyl alcohol n-amino ether, vanillyl alcohol isoamyl ether, vanillyl alcohol n-hexyl ether, vanillyl alcohol methyl ether, vanillyl alcohol ethyl ether, gingerol, shogaol, zingerone, capsaicin, dihydrocapsaicin, nordihydrocapsaicin, homocapsaicin, homodihydrocapsaicin, iso-propyl alcohol, iso-amylalcohol, benzyl alcohol, eugenol, cinnamon oil, cinnamic aldehyde, and mixtures thereof;
UV-filter substances, such as UVA filters, UVB filters and inorganic pigments, preferably, wherein the UV filters are selected from the group consisting of p-aminobenzoic acid, p-aminobenzoic acid ethyl ester (25 mol) ethoxylated, pdimethylaminobenzoic acid 2-ethylhexyl ester, p-aminobenzoic acid ethyl ester (2 mol) Npropoxylated, p-aminobenzoic acid glycerol ester, salicylic acid homomenthyl ester (homosalate) (Neo Heliopan®HMS), salicylic acid 2-ethylhexyl ester (Neo Heliopan®OS), triethanolamine salicylate, 4-isopropylbenzyl salicylate, anthranilic acid menthyl ester (Neo Heliopan®MA), diisopropylcinnamic acid ethyl ester, p-methoxycinnamic acid 2-ethylhexyl ester (Neo Heliopan®AV), diisopropylcinnamic acid methyl ester, pmethoxycinnamic acid isoamyl ester (Neo Heliopan®E 1000), p-methoxycinnamic acid diethanolamine salt, p-methoxycinnamic acid isopropyl ester, 2-ethylhexyl 2-cyano-3,3-diphenylacrylate (Neo Heliopan□303), ethyl 2-cyano-3,3′-diphenylacrylate, 2-phenylbenzimidazolesulfonic acid and salts (Neo Heliopan®Hydro), 3-(4′-trimethylammonium)-benzylidene-bornan-2-one methyl-sulfate, terephthalylidenedibornanesulfonic acid and salts (MexoryKSX), 4-t-butyl-4′-methoxy-dibenzoylmethane (avobenzone)/(Neo Heliopan®357), β-Imidazole-4 (5)-acrylic acid (urocanic acid), 2-hydroxy-4-methoxybenzophenone (Neo Heliopan®BB), 2-hydroxy-4-methoxybenzophenone-5-sulfonic acid, dihydroxy-4-methoxybenzophenone, 2,4-dihydroxybenzophenone, tetrahydroxybenzophenone, 2,2′-dihydroxy-4,4′-dimethoxybenzophenone, 2-hydroxy-4-n-octoxybenzophenone, 2-hydroxy-4-methoxy-4′-10 methylbenzophenone, 3-(4′-sulfo) benzylidene-bornan-2-one and salts, 3-(4′-methylbenzylidene)-d, I-camphor (Neo Heliopan®MBC), 3-benzylidene-d,I-camphor, 4-isopropyldibenzoylmethane, 2,4,6-trianilino-(p-carbo-2′-ethylhexyl-1′-oxy)-1,3,5-triazine, phenylene-bis-benzimidazyl-tetrasulfonic acid disodium salt (Neo Heliopan®AP), 2,2′-(1,4-phenylene)-bis-(1H-benzimidazole-4,6-disulfonic acid), monosodium salt, N-[(2 and 4)-[2-(oxoborn-3-ylidene) methyl]benzyl]-acrylamide polymer, phenol,-(2H-benzotriazol-2-yl)-4-methyl-6-(2-methyl-3 (1,3,3,3-tetramethyl-1-(trimethylsilyl)-oxy)-disiloxyanyl)-propyl), (Mexoryl®XL), 4,4′-[(6-[4-(1, 1-dimethyl)-aminocarbonyl)-phenylamino]-1,3,5-triazine-2,4-diyl) diimino]-bis-(benzoic acid 2-thylhexyl ester) (Uvasorb®HEB), 2,2′-methylene-bis-(6-(2H-benzotriazol-2-yl)-4-(1,1,3,3-tetramethylbutyl)-phenol), (Tinosorb®M), 2,4-bis-[4-(2-ethylhexyloxy)-2-hydroxyphenyl]-1,3,5-triazine, benzylidene malonate-polysiloxane (Parsol®SLX), glyceryl ethylhexanoate dimethoxycinnamate, disodium 2,2′-dihydroxy-4,4′-dimethoxy5,5′-disulfo-benzophenone, dipropylene glycol salicylate, sodium hydroxymethoxybenzophenone-sulfonate, 4,4′,4-(1,3,5-triazine-2,4,6-triyltriimino)-trisbenzoic acid tris (2-ethylhexyl ester) (Uvinul®T150), 2,4-bis-[{(4-(2-ethyl-hexyloxy)-2-hydroxy}phenyl]-6-(4-methoxyphenyl)-1,3,5-triazine, (Tinosorb®S), 2,4-bis-[{(4-(3-sulfonato)-2-hydroxy-propyloxy)-2-hydroxy}-phenyl]-6-(4-methoxyphenyl)-1,3,5-triazine sodium salt, 2,4-bis-[{(3-(2-propyloxy)-2-hydroxy-propyloxy)-2-hydroxy}-phenyl]-6-(4-methoxy-phenyl)-1,3,5-triazine, 2,4-bis-[{4-(2-ethyl-hexyloxy)-2-hydroxy}phenyl]-6-[4-(2-methoxyethyl-carbonyl)-phenylamino]-1,3,5-triazine, 2,4-bis-[{4-(3-(2-propyloxy)-2-hydroxy-propyloxy)-2-hydroxy}-phenyl]-6-[4-(2-ethylcarboxyl)-phenylamino]-1,3,5-triazine, 2,4-bis-[{4-(2-ethyl-hexyloxy)-2-hydroxy}-phenyl]-6-(1-methyl-pyrrol-2-yl)-1,3,5-triazine, 2,4-bis-[{4-tris-(trimethylsiloxy-silylpropyloxy)-2-hydroxy}-phenyl]-6-(4-methoxyphenyl)-1,3,5-triazine, 2,4-bis-[{4-(2″-methylpropenyloxy)-2-hydroxy}-phenyl]-6-(4-methoxyphenyl)-1,3,5-triazine, 2,4-bis-[{4-(1′, 1′, 1′, 3′, 5′, 5′, 5′-heptamethylsiloxy-2″-methyl-propyloxy)-2-hydroxy}-phenyl]-6-(4-methoxyphenyl)-1,3,5-triazine, 2-(4-diethylamino-2-hydroxybenzoyl)-benzoic acid hexyl ester (Uvinuk® A Plus) and indanylidene compounds according to DE 100 55 940 (=WO 02/38537); and/or preferably wherein the pigments are selected from the group consisting of metal oxides and metal salts, for example titanium dioxides, zinc oxide (ZnO), iron oxides (e.g. Fe2O3), aluminium oxide (Al2O3); cerium oxides (e.g. Ce2O3), manganese oxides (e.g. MnO), zirconium oxide 30 (ZrO2), silicon oxide (SiO2), mixed oxides of the corresponding metals and mixtures of such oxides, barium sulfate and zinc stearate, particularly preferably pigments based on TiO2 or zinc oxide, further preferably coated titanium dioxides, such as e.g. titanium dioxide T 805 (Degussa) or Eusolex® T2000 (Merck), or coated zinc oxide, such as e.g. Zinc Oxide NDM. (In this context, possible hydrophobic coating agents may be, for example, silicones, particularly trialkoxyoctysilanes or simethicone)

Carriers, auxiliary substances and additional substances (excluding water) can generally be included in a cosmetic composition according to the invention in quantities of 1 to 95 wt. %, preferably 5 to 70 wt. %, more preferably 5 to 50 wt. %, in each case based on the total weight of the composition. The amounts of such carriers, auxiliary substances and additional substances to be used in each case can easily be determined by the person skilled in the art by simple trials, depending on the nature of the particular composition.

A cosmetic composition according to the invention may be selected from the group consisting of cosmetic products for treatment, protecting, care and cleansing of the skin and/or hair or make-up products, preferably leave-on products, more preferably in the form or selected from the product group consisting of an alcoholic or aqueous/alcoholic solution, dispersion, suspension, emulsion (preferably cream, lotion or milk of the W/O, O/W or multiple emulsion, PIT emulsion, emulsion foam, micro-, nanoemulsion, Pickering emulsion type), ointment, paste, gel (preferably hydro-, hydrodispersion-, oleogel), balm, serum, powder, wipe, Eau de Toilette, Eau de Cologne, perfume, stick, roll-on, (pump) spray, aerosol, leave-on skin care composition (preferably face-care composition), leave-on insect repellent composition, sunscreen composition, skin-lightening composition, self-tanning composition, aftersun preparation, shaving or after-shave composition, hair-removing composition, hair care composition, preferably conditioner, hair lotion, hair tonic, styling cream, pomade, styling aid (preferably gel or wax), permanent wave and fixing compositions, hair smoothing composition (straightening composition, relaxer), hair setting composition, blonding composition, hair colouring composition, such as e.g. temporary, directly absorbed, semi-permanent hair colouring composition, permanent hair colouring composition, decorative cosmetic composition (preferably face powder, eye shadow, kajal pencil, lipstick), deodorant and/or antiperspirant composition.

Preferably, none of the additives of a composition according to the invention comprises dicaffeoylquinic acids or tocopherol, particularly preferably 3,4-dicaffeoylquinic acid, 3,5-dicaffeoylquinic acid, 1,5-dicaffeoylquinic acid or 4,5-dicaffeoylquinic acid or tocopherol, in addition to the dicaffeoylquinic acids or tocopherol provided as component (a) or (b).

Preferably, in case the additives of a composition according to the invention comprises dicaffeoylquinic acids or tocopherol, particularly preferably 3,4-dicaffeoylquinic acid, 3,5-dicaffeoylquinic acid, 1,5-dicaffeoylquinic acid or 4,5-dicaffeoylquinic acid or tocopherol, the respective amounts of the substances of the additives are also considered when the (total) weight of these substances is to be determined.

A composition for food or pleasure may preferably be understood as a composition selected from the group consisting of may be selected from the group consisting of (reduced-calorie) baked goods (e.g. bread, dry biscuits, cakes, other baked articles), confectionery (e.g. muesli bar products, chocolates, chocolate bars, other products in bar form, fruit gums, dragees, hard and soft caramels, chewing gum), non-alcoholic drinks (e.g. cocoa, coffee, green tea, black tea, (green, black) tea drinks enriched with (green, black) tea extracts, rooibos tea, other herbal teas, fruit-containing soft drinks, isotonic drinks, refreshing drinks, nectars, fruit and vegetable juices, fruit or vegetable juice preparations), instant drinks (e.g. instant cocoa drinks, instant tea drinks, instant coffee drinks), meat products (e.g. ham, fresh sausage or raw sausage preparations, spiced or marinated fresh or salt meat products), eggs or egg products (dried egg, egg white, egg yolk), cereal products (e.g. breakfast cereals, muesli bars, precooked ready-to-eat rice products), dairy products (e.g. full-fat or reduced-fat or fat-free milk drinks, rice pudding, yoghurt, kefir, cream cheese, soft cheese, hard cheese, dried milk powder, whey, butter, buttermilk, ice-cream, partially or completely hydrolysed milk-protein-containing products), products made from soy protein or other soybean fractions (e.g. soy milk and products produced therefrom, drinks containing isolated or enzymatically treated soy protein, drinks containing soy flour, preparations containing soy lecithin, fermented products such as tofu or tempeh or products produced therefrom and mixtures with fruit preparations and optionally flavours), dairy-like preparations (milk-type, yoghurt-type, dessert-type, ice cream) from protein rich plant materials (e.g. from seed materials of oat, almond, pea, lupine, lentils, faba beans, chickpea, rice, canola), plant protein-enriched non-dairy drinks, fruit preparations (e.g. jams, sorbets, fruit sauces, fruit fillings), vegetable preparations (e.g. ketchup, sauces, dried vegetables, frozen vegetables, precooked vegetables, boiled-down vegetables), snacks (e.g. baked or fried potato crisps or potato dough products, maize-or groundnut-based extrudates), fat- and oil-based products or emulsions thereof (e.g. mayonnaise, remoulade, dressings, in each case full-fat or reduced-fat), other ready-made dishes and soups (e.g. dried soups, instant soups, precooked soups), spices, spice mixtures and in particular seasonings which are used, for example, in the snacks field, sweetener preparations, tablets or sachets, other preparations for sweetening or whitening drinks.

A pharmaceutical composition according to the invention is preferably suitable for topical application to the skin of a human being or animal to achieve a pharmaceutical effect. A pharmaceutical composition according to the invention may preferably comprise carriers, auxiliary substances and additional substances. Preferably, these carriers, auxiliary substances and additional substances can be selected from those described herein for the cosmetic composition according to the invention.

It is particularly preferred in a composition according to the invention that one or more UV filters, preferably as described above, is/are present. UV filters reduce, inhibit or prevent the formation of reactive oxygen species (ROS) and thus reduce, inhibit or prevent damages in cells, such as protein damages.

The composition according to the invention comprises a mixture according to the invention, which comprises components (a) and (b) in a particular weight ratio. However, it may be possible that the composition comprises further ingredients, which may also include a compound covered by component (a) and/or (b). However, it is preferred that in the composition according to the invention, the weight ratio of components (a) and (b) is in a comparable range as in the mixture according to the invention.

Therefore, it is preferred in a composition according to the invention, that the weight ratio of the total weight of component (a) in the composition to the total weight of component (b) in the composition is in a range of from 1:1000 to 5:1, preferably in a range of from 1:800 to 2:1, particularly preferably in a range of from 1:600 to 1:1, especially preferably in a range of from 1:500 to 1:2, more preferably in a range of from 1:300 to 1:5, further preferably in a range of from 1:250 to 1:10; especially preferably in a range of from 1:100 to 1:20 even more preferably in a range of from 1:75 to 1:25. Furthermore in a composition according to the invention, the ratio described above may be any combination of any upper and any lower limit or any upper and any other upper limit or any lower and any other lower limit, as described above.

Moreover, the invention relates to a pharmaceutical composition according to the invention for use as a medicament.

As described above, it was surprisingly found that a composition according to the invention may be used for (synergistically) stimulating the expression of one or more heat shock proteins (HSP).

Furthermore, in this regard, the pharmaceutical composition according to the invention may particularly advantageously be used in treating or preventing one or more diseases or unhealthy conditions of human skin, in particular skin aging and/or increasing the thermotolerance of the skin of a subject and/or increasing the resistance of the skin or skin cells cells to low humidity and/or improving the barrier function of keratinocytes and/or increasing the skin moisture of a subject.

Therefore, the invention relates to a pharmaceutical composition according to the invention for use in the treatment and/or prevention of one or more diseases or unhealthy conditions of human skin, in particular skin aging.

Preferably, the treatment and/or prevention comprises or consists of a topical application of the pharmaceutical composition according to the invention.

As described above, it has been surprisingly found that dicaffeoylquinic acid(s) are able to induce the expression of one or more heat shock proteins (HSP), whereas also surprisingly, monocaffeoylquinic acids were not able to induce HSPs.

Thus, the invention further relates to a non-therapeutic use of one or more dicaffeoylquinic acid(s),
preferably of 3,4-dicaffeoylquinic acid, 3,5-dicaffeoylquinic acid, 1,5-dicaffeoylquinic acid, 4,5-dicaffeoylquinic acid or mixtures thereof,
for stimulating the expression of one or more heat shock proteins (HSP), preferably in keratinocytes.

As also described above, skin aging is amongst others characterized by a decreased number of chaperones and caused or, respectively, triggered by factors such as heat exposure of the skin.

HSPs are involved in chaperoning proper protein folding and in regulating the thermotolerance of a subject, as described above. Thus, the invention further relates to a cosmetic, non-therapeutic use of one or more dicaffeoylquinic acid(s),
preferably of 3,4-dicaffeoylquinic acid, 3,5-dicaffeoylquinic acid, 1,5-dicaffeoylquinic acid, 4,5-dicaffeoylquinic acid or mixtures thereof,
for reducing or preventing skin aging in a subject and/or for increasing the thermotolerance of the skin of a subject and/or for increasing the resistance of the skin or skin cells to low humidity.

Moreover and as described above, it has been surprisingly found that dicaffeoylquinic acid(s) are able to induce the expression of genes encoding “mechanical”, desmosomal and tight junctional proteins, whereas also surprisingly, monocaffeoylquinic acids were not able to induce such genes.

Additionally and as also described above, an improved barrier function of keratinocytes leads to a reduced loss of water of the skin. Consequently, an improved barrier function will result in less water loss of the skin and thus to an overall increase in skin moisture of a subject.

Thus, the invention further relates to a non-therapeutic use of one or more dicaffeoylquinic acid(s),
preferably of 3,4-dicaffeoylquinic acid, 3,5-dicaffeoylquinic acid, 1,5-dicaffeoylquinic acid, 4,5-dicaffeoylquinic acid or mixtures thereof,
for improving the barrier function of keratinocytes and/or for increasing the skin moisture of a subject.

In addition to the above surprising findings, it was even more surprisingly found that tocopherol synergistically acts with dicaffeoylquinic acid in achieving the above effects.

The term “synergistically” as used herein describes the extent of an effect of a combination of two or more substances, which is beyond the mere additive effect of the single substances, i.e. a stronger effect is achieved. This definition is well known to the skilled person.

Consequently, the invention also relates to a non-therapeutic use of a mixture or of a composition comprising or consisting of
(a) one or more dicaffeoylquinic acid(s),
   preferably selected from the group consisting of 3,4-dicaffeoylquinic acid, 3,5-dicaffeoylquinic acid, 1,5-dicaffeoylquinic acid, 4,5-dicaffeoylquinic acid or mixtures thereof, and
(b) tocopherol,
   preferably of a mixture according to the invention or of a composition according to the invention for stimulating the expression of one or more heat shock proteins (HSP), preferably in keratinocytes, preferably for synergistically stimulating the expression of one or more heat shock proteins (HSP), preferably in keratinocytes.

Furthermore, the invention relates to a non-therapeutic use of a mixture or of a composition comprising or consisting of
(a) one or more dicaffeoylquinic acid(s),
   preferably selected from the group consisting of 3,4-dicaffeoylquinic acid, 3,5-dicaffeoylquinic acid, 1,5-dicaffeoylquinic acid, 4,5-dicaffeoylquinic acid or mixtures thereof, and
(b) tocopherol,
   preferably of a mixture according to the invention or of a composition according to the invention,
   for reducing or preventing skin aging in a subject and/or for increasing the thermotolerance of the skin of a subject and/or for increasing the resistance of the skin or skin cells to low humidity, preferably for synergistically reducing or preventing skin aging in a subject and/or for synergistically increasing the thermotolerance of the skin of a subject and/or for synergistically increasing the resistance of the skin or skin cells to low humidity.

Additionally, the invention relates to a non-therapeutic use of a mixture or of a composition comprising or consisting of
(a) one or more dicaffeoylquinic acid(s),
   preferably selected from the group consisting of 3,4-dicaffeoylquinic acid, 3,5-dicaffeoylquinic acid, 1,5-dicaffeoylquinic acid, 4,5-dicaffeoylquinic acid or mixtures thereof, and
(b) tocopherol,
   preferably of a mixture according to the invention or of a composition according to the invention, for improving the barrier function of keratinocytes and/or for increasing the skin moisture of a subject, preferably for synergistically improving the barrier function of keratinocytes and/or preferably for synergistically increasing the skin moisture of a subject.

Likewise, the invention relates to methods for stimulating the expression of one or more heat shock proteins (HSP), preferably in keratinocytes, for reducing or preventing skin aging in a subject, for increasing the thermotolerance of the skin of a subject, for increasing the resistance of the skin or skin cells to low humidity, for improving the barrier function of keratinocytes or for increasing the skin moisture of a subject, comprising or consisting of the step
administering one or more dicaffeoylquinic acid(s), preferably 3,4-dicaffeoylquinic acid, 3,5-dicaffeoylquinic acid, 1,5-dicaffeoylquinic acid, 4,5-dicaffeoylquinic acid or a mixture thereof, to the skin of a subject.

Additionally, the invention relates to methods for synergistically stimulating the expression of one or more heat shock proteins (HSP), preferably in keratinocytes, for synergistically reducing or preventing skin aging in a subject, for synergistically increasing the thermotolerance of the skin of a subject, for synergistically increasing the resistance of the skin or skin cells to low humidity, for synergistically improving the barrier function of keratinocytes, or for synergistically increasing the skin moisture of a subject, comprising or consisting of the step
administering
   one or more dicaffeoylquinic acid(s), preferably 3,4-dicaffeoylquinic acid, 3,5-dicaffeoylquinic acid, 1,5-dicaffeoylquinic acid, 4,5-dicaffeoylquinic acid or mixtures thereof, and
   tocopherol
   to the skin of a subject, preferably wherein the administration is a simultaneous administration (e.g. the components are administered in a mixture comprising or consisting of these components)
   and/or
administering a mixture according to the invention and/or a composition according to the invention
   to the skin of a subject.

The term “subject”, as used herein, describes a human or an animal. Preferably, when the treatment of a subject is described herein, the subject is a human or animal in need of such a treatment.

The term “skin”, as used herein, preferably describes cutaneous skin of the body, face, scalp, oral and/or nail bed skin.

The term “keratinocytes”, as used herein, describes various keratinocytes, such as epidermal keratinocytes, oral keratinocytes, hair follicular keratinocytes (e.g. matrix cells and outer root sheath keratinocytes) and nail keratinocytes. Preferably, the term “keratinocytes” refers to keratinocytes in any stage of differentiation and thus also includes proliferating and non-proliferating keratinocytes.

The term “improving the barrier function of keratinocytes”, as used herein, describes an improvement of the epidermal barrier, which is crucial to prevent entry of noxious stimuli. Furthermore, the term describes that, on a structural level, the tight mechanical cohesions between the cells of the same and different epidermal layers (e.g. tight junctions, (corneo) desmosomes and adherens junctions) are strengthened or increased in their amount. The skin barrier function is well known to a skilled person and may be determined by various methods, such as by transepithelial electrical resistance (TEER).

The term “reducing or preventing skin aging in a subject”, as used herein, describes a deceleration or prevention of the formation of new fine lines and wrinkles and/or of the size and depth of already present wrinkles. Additionally or alternatively, the term describes a deceleration or prevention of the loss of elasticity of the skin. The number, size and depth of wrinkles can be easily determined e.g. by optical means such as counting or measuring (particularly the size). The elasticity of the skin can also be determined by established methods such as measuring with a cutometer.

The term “increasing the skin moisture of a subject”, as used herein, describes a deceleration or prevention of loss of skin moisture such as to the surrounding air. The particular skin moisture can be determined by established methods such as measuring by corneometry or measuring the transepidermal water loss.

The term “increasing thermotolerance”, as used herein, describes any improvement with regard to the thermotolerance of a cell, particularly a keratinocyte, i.e. the tolerance to thermal damage after prior heat or cold conditioning. Thus, when the thermotolerance is increased, within the meaning of the term as used herein, the thermal damage in a cell, preferably a keratinocyte, caused by prior heat or cold conditioning, is reduced.

The term “increasing the resistance of the skin or skin cells to low humidity” describes any cosmetic improvement with regard to a dry appearance of the skin. Since low humidity even increases the loss of water in the skin, skin cells suffer from osmotic stress. However, the suffering is ameliorated by HSPs, particularly by their chaperone function. An increase in the resistance of the skin or skin cells to low humidity can thus be measured by optical evaluation of a dry appearance of the skin.

The term “low humidity” describes the humidity of the surrounding air in a value of less than 30%, preferably less than 25%, particularly preferably less than 20%, relative humidity.

The terms “water in the skin” or “skin moisture” or “skin hydration” describe the intra- and extracellular water in the skin, which can be determined by established methods such as measuring by corneometry.

Precisely, it is shown in the subsequent Examples, that several genes are induced by dicaffeoylquinic acids or synergistically induced by dicaffeoylquinic acids and tocopherol. Thus, it is preferred in the uses according to the invention that the expression of one or more genes selected from the group consisting of HSPB2, HSPB8, CRYAB, CLDN 1, OCLN, DEFB1, DSC1 and DSG1 is induced. I.e. the uses or methods are directed to inducing the expression of one or more genes selected from the group consisting of HSPB2, HSPB8, CRYAB, CLDN1, OCLN, DEFB1, DSC1 and DSG1.

Particularly, the uses or methods directed to stimulating the expression of one or more heat shock proteins (HSP), preferably in keratinocytes, and to increasing the thermotolerance of the skin of a subject are preferably further directed to inducing the expression of one or more genes selected from the group consisting of HSPB2, HSPB8 and CRYAB.

Particularly, the uses or methods directed to improving the barrier function of keratinocytes are preferably further directed to inducing the expression of one or more genes selected from the group consisting of CLDN1, OCLN, DEFB1, DSC1 and DSG1.

Particularly, the uses or methods directed to reducing or preventing skin aging in a subject are preferably further directed to inducing the expression of one or more genes selected from the group consisting of HSPB2, HSPB8, CRYAB, CLDN1, OCLN, DEFB1, DSC1 and DSG1. As described above, skin aging is associated with a decreased number of chaperones, exposure of the skin to heat and a loss of water in the skin. Thus, inducing the expression of one or more genes belonging to the group of HSPs or of genes involved in providing a proper skin barrier is associated with the reduction or prevention of skin aging.

Moreover, it was surprisingly found that tocopherol is able to stabilize dicaffeoylquinic acids in mixtures or composition, whereas other conventional stabilizers did not or not as efficiently provide such and effect, as shown in Example 9.

Therefore, the invention also relates to a use of tocopherol for increasing the stability of one or more dicaffeoylquinic acid(s),
preferably selected from the group consisting of 3,4-dicaffeoylquinic acid, 3,5-dicaffeoylquinic acid, 1,5-dicaffeoylquinic acid, 4,5-dicaffeoylquinic acid or mixtures thereof,
in a mixture or composition, preferably in a mixture according to the invention or in a composition according to the invention.

It is to be noted that this effect provides the advantage that the dicaffeoylquinic acids are particularly stable in the provided mixture or composition. Thus, it can be advantageously made use of the effects of the dicaffeoylquinic acids as described herein as well as of the synergism provided by tocopherol and the dicaffeoylquinic acids as described herein.

Preferred embodiments and further aspects of the present invention emerge from the attached patent claims and the following examples, the examples not being intended to limit the scope of the present invention.

### EXAMPLES

### Example 1

### Modulation of Gene Expression of Keratinocytes by Dicaffeoylquinic Acids

1,5-dicaffeoylquinic acid (CAS number 19870-46-3, synonym: 1,3-dicaffeoylquinic acid), 3,4-dicaffeoylquinic acid (CAS number 57378-72-0, synonym: 4,5-dicaffeoylquinic acid, isochlorogenic acid C), 3,5-dicaffeoylquinic acid (CAS number 2450-53-5, synonym: isochlorogenic acid A) and 4,5-dicaffeoylquinic acid (CAS number 14534-61-3, synonym: 3,4-dicaffeoylquinic acid, isochlorogenic acid B) were bought from Phytolab GmbH & Co. KG, Germany.

Neonatal human epidermal keratinocytes (nHEK) were cultivated in an EpiLife® medium (Gibco) including an HKGS kit (Gibco) with 5% CO2 at 37° C. in accordance with the supplier's instructions. The cells were treated for 24 hours, with 0.0005% of the different dicaffeoylquinic acids from above dissolved in DMSO and DMSO alone as the vehicle control. Genomic target expression levels in treated cells were measured using a quantitative Real-Time PCR comparison to vehicle control treatment.

RNA was isolated using Qiagen's RNeasy® Mini Kit. The total RNA concentrations were measured using Eppendorf's μCuvetteG 1.0 and BioPhotometer, by measuring the absorption at 260 nm. Purity control values such as E260/280 and E260/230 were calculated simultaneously. Reverse transcription was performed using the high-capacity RNA-to-cDNA kit of Applied Biosystems, in accordance with the supplier's instructions. Samples were treated in Biometra's PCR Thermocycler. For fast real-time qPCR, the cDNA was diluted with RNase-free water, and the TaqMan™ Fast Universal PCR Master Mix of Applied Biosystems was used. Quantitative real time PCR was performed using the StepOnePlus fast real-time PCR instrument by Applied Biosystems. Analysis was conducted using the StepOne software and 2-AAct method (normalised to endogenous control HTRP1 expression). For upregulations, RQ values ≥2.5 are considered to be relevant.

TABLE 1
Results

| | RQ value | | | |
|---|---|---|---|---|
| | 0.0005% | 0.0005% | 0.0005% | 0.0005% |
| | 1,5-Di- | 3,4-Di- | 3,5-Di- | 4,5-Di- |
| | caffeoyl- | caffeoyl- | caffeoyl- | caffeoyl- |
| Gene | quinic acid | quinic acid | quinic acid | quinic acid |
| HPRT1 | 1.0 | 1.0 | 1.0 | 1.0 |
| (house- | | | | |
| keeping) | | | | |
| HSPB2 | 2.4 | 4.9 | 6.1 | 4.3 |
| HSPB8 | 2.8 | 7.3 | 6.2 | 4.2 |
| CRYAB | 7.2 | 18.7 | 16.5 | 13.8 |
| CLDN1 | 2.4 | 5.1 | 4.7 | 3.5 |
| OCLN | 2.2 | 4.2 | 3.5 | 2.4 |
| DEFB1 | 2.4 | 3.5 | 3.4 | 3.2 |
| DSC1 | 7.6 | 25.8 | 20.5 | 18.1 |
| DSG1 | 5.3 | 14.9 | 12.2 | 10.6 |

### Example 2

### Modulation of Gene Expression of Keratinocytes by Monocaffeoylquinic Acids

3-Caffeoylquinic acid (CAS number 327-97-9, synonym: chlorogenic acid, Sigma-Aldrich), 5 4-caffeoylquinic acid (CAS number 905-99-7, synonym: cryptochlorogenic acid, Phytolab GmbH & Co. KG) and 5-caffeoylquinic acid (CAS number 906-33-2, synonym: neochlorogenic acid, Phytolab GmbH & Co. KG) were purchased.

Neonatal human epidermal keratinocytes (nHEK) were cultivated as described in Example 1 and treated for 24 hours with 0.0007% of the different monocaffeoylquinic acids. Afterwards fast real-time qPCR was performed as described in above using a customised gene array.

For upregulations, RQ values ≥2.5 are considered to be relevant.

TABLE 2
Results

| | RQ value | | |
|---|---|---|---|
| | 0.0007% | 0.0007% | 0.0007% |
| | 3-caffeoyl-quinic | 4-caffeoyl-quinic | 5-caffeoyl-quinic |
| Gene | acid | acid | acid |
| HPRT1 | 1.0 | 1.0 | 1.0 |
| (housekeeping) | | | |
| HSPB2 | 0.8 | 2.2 | 1.0 |
| HSPB8 | 0.8 | 1.0 | 0.6 |
| CRYAB | 0.9 | 1.9 | 0.7 |
| CLDN1 | 0.8 | 0.8 | 0.8 |
| OCLN | 1.0 | 1.0 | 0.8 |
| DEFB1 | 1.1 | 1.9 | 0.7 |
| DSC1 | 1.0 | 2.2 | 0.7 |
| DSG1 | 0.7 | 1.9 | 0.5 |

The results show that surprisingly none of the tested monocaffeoylquinic acids relevantly modulates any of the analyzed genes.

### Example 3

### Effect of Dicaffeoylquinic Acid Containing Plant Extracts on the Modulation of Gene Expression of Keratinocytes

To investigate if dicaffeoylquinic acid containing plant extracts also exhibit the observed gene modulating effect, yarrow (Achillea millefolium, freeze-dried fresh press juice of flowering aerials, cultivated in Germany), arnica flower (Arnica montana blossom water extract 55.8%, glucose syrup 44.0%, 0.2% potassium sorbate, dry matter 69.4%, collected in East Europe), elderflower (Sambus nigra flowerwater extract 80.8%, glucose syrup 19.0%, 0.2% potassium sorbate, dry matter 65.0%, collected in East Europe), artichoke (Cynara Scolymus concentrated pressed juice 80%, glucose syrup 20%, 52-55° Brix, cultivated in Spain), ivy leaf (Hedera helix leaf water extract 62.3%, glucose syrup 37.50%, 0.2% potassium sorbate, 55-65° Brix, collected in East Europe), honeysuckle flower (Lonicera japonica water extract powder, loss on drying <5%, cultivated in China), Chrysanthemum indica flower (water extract powder, loss on drying <5%, cultivated in China), and green coffee bean (Coffea robusta seed extract 70%, maltodextrin 30%, moisture content: 5.2%, cultivated in Brazil, Columbia, Mexico) extract were tested. Furthermore, commercial extracts of ivy, honeysuckle and chrysanthemum were tested.

The following commercial extracts were used:
Extrapone® Ivy, which contains an ivy leaf (Hedera helix) water extract prepared in propylene glycol and water (collected in Eastern Europe),
Actipone® Honeysuckle, which contains the water extract of Lonicera japonica flowers prepared in water and propylene glycol (cultivated in China),
Extrapone® Chrysanthemum, which contains the extract of Chrysanthemum indica flowers prepared in water and propylene glycol (cultivated in China).

Extracts were characterized by HPLC analysis:
HPLC method:
Column: YMC ODS-AQ, 5 μm, 150×3 mm+10×3 mm
Temperature: 40° C.
Flow. 0.6 ml/min
Solvent A: Water+0.1% formic acid
Solvent B: Acetonitrile+0.1% formic acid

| | | | |
|---|---|---|---|
| Gradient: | Time [min] | % A | % B |

| | | |
|---|---|---|
| 0 | 90 | 10 |
| 15.0 | 70 | 30 |
| 20.0 | 50 | 50 |
| 24.0 | 0 | 100 |
| 25.0 | 0 | 100 |

Injection Volume: 5.0 μl
DAD Signal: 330 nm

TABLE 3
Analytical results

| | Content by HPLC [%] | | | |
|---|---|---|---|---|
| | Sum of | | | Di- |
| | 1,5-and | | | caffeoyl- |
| | 3,5-Di- | 4,5-Di- | 3,4-Di- | quinic |
| | caffeoyl- | caffeoyl- | caffeoyl- | acids in |
| Extract | quinic acid* | quinic acid | quinic acid | sum |
| Yarrow Extract | 0.47 | 0.57 | 0.54 | 1.58 |
| Arnica flower | 0.47 | 0.13 | 0.03 | 0.65 |
| Extract | | | | |
| Elderflower | 0.07 | 0.06 | 0.02 | 0.14 |
| Extract | | | | |
| Ivy leaf Extract | 0.035 | 0.028 | 0.025 | 0.087 |
| Extrapone ® | 0.003 | 0.003 | 0.002 | 0.008 |
| Ivy | | | | |
| Honeysuckle | 0.51 | 1.20 | 0.35 | 2.06 |
| flower Extract | | | | |
| Actipone ® | 0.002 | 0.003 | 0.002 | 0.007 |
| Honeysuckle | | | | |
| Chrysanthemum | 0.27 | 0.41 | 0.35 | 1.03 |
| Extract | | | | |
| Extrapone ® | 0.008 | 0.013 | 0.010 | 0.031 |
| Chrysanthemum | | | | |
| Artichoke | 0.54 | 0.18 | 0.18 | 0.90 |
| Extract | | | | |
| Green coffee | 0.76 | 0.91 | 0.08 | 1.75 |
| bean Extract | | | | |

*1,5- and 3,5-Dicaffeoyl-quinic acid exhibited overlaying peaks and were not sufficiently separated by the used HPLC method and are therefore given as sum.

Neonatal human epidermal keratinocytes (nHEK) were cultivated as described in Example 1 and treated for 24 hours with 0.005% of the extract samples. Afterwards fast real-time qPCR was performed as described in above using a customized gene array.

For upregulations, RQ values ≥2.5 are considered to be relevant.

TABLE 4
Results

| | RQ value | | |
|---|---|---|---|
| | 0.005% | 0.005% | 0.005% |
| Gene | Yarrow extract | Arnica extract | Green coffee extract |
| HPRT1 | 1.0 | 1.0 | 1.0 |
| (housekeeping) | | | |
| HSPB8 | 4.6 | 1.7 | 1.4 |
| CRYAB | 14.0 | 3.9 | 3.8 |
| CLDN1 | 3.1 | 1.6 | 1.6 |
| OCLN | 2.8 | 1.1 | 1.1 |
| DSC1 | 20.4 | 3.5 | 3.1 |
| DCG1 | 8.2 | 2.7 | 3.4 |

The results show that dicaffeoylquinic acids exhibit their gene-modulating efficacy also when contained in plant extract.

Furthermore, comparable results were obtained for the extracts of elderflower, ivy, honeysuckle, chrysanthemum and artichoke.

### Example 4

### Synergistic Effect of 4,5-Dicaffeoylquinic Acid and Tocopherol on the Modulation of Gene Expression of Keratinocytes

Neonatal human epidermal keratinocytes (nHEK) were cultivated as described in Example 1 and treated for 24 hours with 4,5-dicaffeoylquinic acid (CAS number 14534-61-3, Phytolab GmbH & Co. KG) and (±)-α-tocopherol (CAS number 10191-41-0, synonym: Synonym: DL-α-Tocopherol, Vitamin E, Sigma-Aldrich, synthetic) alone and in combination. Afterwards fast real-time qPCR was performed as described in above using a customised gene array.

For upregulations, RQ values ≥2.5 are considered to be relevant.

Kull's equation for calculation of the synergism index SI was used:

$$SI = CxD / A + CxE / B$$

With
A=RQ value by 4,5-dicaffeoylquinic acid at concentration x
B=RQ value by tocopherol at concentration y
C=RQ value by the combination of 4,5-dicaffeoylquinic acid at concentration x/2 and tocopherol at concentration y/2
D=Factor for 4,5-dicaffeoylquinic acid=>0.5 (due to half concentration tested in the combination)
E=Factor for tocopherol=>0.5 (due to half concentration tested in the combination)
A SI=1 is obtained for additive activity of the two combined components, whereas a SI <1 proves antagonistic activity (observed efficacy is lower than additive) and SI >1 proves synergistic activity (observed efficacy is higher than additive). Results of this experiment are summarized in Table 5

TABLE 5
Results

| | RQ value | | | |
|---|---|---|---|---|
| | 0.0007% | 0.00035% | | |
| | 4,5-di- | 4,5-di-caffeoyl- | 0.005% | |
| | caffeoyl- | quinic acid + | (±)- | Synergy |
| | quinic | 0.0025% (±)- | alpha- | Index |
| Gene | acid | alpha-tocopherol | tocopherol | SI |
| HPRT1 (house- | 1.0 | 1.0 | 1.0 | |
| keeping) | | | | |
| HSPB2 | 5.7 | 6.4 | 1.4 | 4.2 |
| HSPB8 | 6.2 | 5.4 | 1.0 | 4.6 |
| CRYAB | 21.1 | 20.5 | 1.4 | 13.1 |
| CLDN1 | 2.4 | 3.1 | 1.2 | 2.4 |
| OCLN | 3.4 | 3.9 | 1.7 | 2.1 |
| DEFB1 | 4.3 | 3.9 | 1.3 | 2.7 |
| DSC1 | 25.0 | 24.7 | 1.1 | 19.9 |
| DSG1 | 11.1 | 11.0 | 1.3 | 7.6 |

The results clearly show that surprisingly a combination of 4,5-di-caffeoylquinic acid and (±)-alpha-tocopherol relevantly and furthermore also synergistically up-regulates HSPB2, HSPB8, CRYAB, CLDN1, OCLN, DEFB1, DSC1 and DSG1 genes. The observed synergistic effect was most pronounced (SI >5) for CRYAB, DSC1 and DSG1.

(±)-Alpha-tocopherol alone when tested at 0.005% does not relevantly modulate any of the selected genes.

The tested combination (test concentration 0.00285%) is composed of 12.3% 4,5-dicaffeoylquinic acid (test concentration of 0.00035% in the combination) and 87.7% (±)alpha-tocopherol (test concentration of 0.0025% in the combination).

### Example 5

### Synergistic Effect of 3,4-Dicaffeoylquinic Acid and Tocopherol on the Modulation of Gene Expression of Keratinocytes

Neonatal human epidermal keratinocytes (nHEK) were cultivated as described in Example 1 and treated for 24 hours with 3,4-dicaffeoylquinic acid (CAS number 57378-72-0, Phytolab GmbH & Co. KG) and (+)-α-tocopherol (CAS number 59-02-9, synonym: Synonym: D-α-tocopherol, Vitamin E, Sigma-Aldrich) alone and in combination. Afterwards fast real-time qPCR was performed as described in above using a customised gene array. For upregulations, RQ values ≥2.5 are considered to be relevant.

### Combination A:

The tested combination A (test concentration 0.0018%) is composed of 16.7% 3,4-dicaffeoyl quinic acid (test concentration of 0.0003% in the combination) and 83.1% (+)alpha-tocopherol (test concentration of 0.0015% in the combination).

Kull's equation was used for the calculation of the synergism index SI as described in example 4:

TABLE 6
Results of combination A

| | RQ value | | | |
|---|---|---|---|---|
| | 0.0006% | 0.0003% | | |
| | 3,4-di- | 3,4-di-caffeoyl- | 0.003% | |
| | caffeoyl- | quinic acid + | (+)- | Synergy |
| | quinic | 0.0015% (+)- | alpha- | Index |
| Gene | acid | alpha-tocopherol | tocopherol | SI |
| HPRT1 | 1.0 | 1.0 | 1.0 | |
| (housekeeping) | | | | |
| CLDN1 | 4.2 | 3.3 | 0.4 | 4.0 |
| DSC1 | 6.2 | 11.0 | 0.6 | 9.6 |
| DSG1 | 2.1 | 8.9 | 0.4 | 11.9 |

The results clearly show that surprisingly combination A of 3,4 di-caffeoylquinic acid and (+)-alpha-tocopherol relevantly and furthermore also synergistically up-regulates CLDN1, DSC1 and DSG1 genes.

(+)-Alpha-tocopherol alone when tested at 0.003% does not relevantly modulate any of the selected genes.

### Combination B:

Combination B (test concentration 0.0022%) is composed of 9.1% 3,4-di-caffeoylquinic acid (test concentration of 0.0002% in the combination) and 90.9% (+)-alpha-tocopherol (test concentration of 0.002% in the combination).

Kull's equation was also used for the calculation of the synergism index SI of combination B:

$$SI = CxD / A + CxE / B$$

With

A=RQ value by 3,4-dicaffeoylquinic acid at concentration x

B=RQ value by tocopherol at concentration y

C=RQ value by the combination of 3,4-dicaffeoylquinic acid at concentration x/3 and tocopherol at concentration 2y/3

D=Factor for 3,4-dicaffeoylquinic acid=>0.3333 (due to x/3 concentration tested in the combination)

E=Factor for tocopherol=>0.6667 (due to 2y/3 concentration tested in the combination)

TABLE 7
Results combination B

| | RQ value | | | |
|---|---|---|---|---|
| | 0.0006% | 0.0002% | | |
| | 3,4-di- | 3,4-di-caffeoyl- | 0.003% | |
| | caffeoyl- | quinic acid + | (+)- | Synergy |
| | quinic | 0.002% (+)- | alpha- | Index |
| Gene | acid | alpha-tocopherol | tocopherol | SI |
| HPRT1 | 1.0 | 1.0 | 1.0 | |
| (housekeeping) | | | | |
| CLDN1 | 4.2 | 5.7 | 0.4 | 7.6 |
| DSC1 | 6.2 | 24.7 | 0.6 | 27.1 |
| DSG1 | 2.1 | 8.7 | 0.4 | 11.6 |

The results clearly show that surprisingly also combination b of 3,4-di-caffeoylquinic acid and (+)-alpha-tocopherol relevantly and furthermore also synergistically up-regulates CLDN1, DSC1 and DSG1 genes. The observed synergistic effect was very pronounced (SI >5).

(+)-Alpha-tocopherol alone when tested at 0.003% does not relevantly modulate any of the selected genes.

### Example 6

### Synergistic Effect of 3,5-Dicaffeoylquinic Acid and Tocopherol on the Modulation of Gene Expression of Keratinocytes

Neonatal human epidermal keratinocytes (nHEK) were cultivated as described above and treated for 24 hours with 3,5-dicaffeoylquinic acid (CAS number 2450-53-5, Phytolab GmbH & Co. KG) and D-α-tocopherol (CAS number 59-02-9, synonym: (+)-α-tocopherol, Vitamin E, trade name: Copherol® F 1300 C, BTC Europe GmbH) alone and in combination. Afterwards fast real-time qPCR was performed as described in above using a customised gene array.

For upregulations, RQ values ≥2.5 are considered to be relevant.

The tested combinations (test concentration 0.0051%) is composed of 2.0% 3,5-dicaffeoylquinic acid (test concentration of 0.0001 in the combination) and 98.0% (+)-alpha-tocopherol (test concentration of 0.005% in the combination).

Kull's equation was used for the calculation of the synergism index SI as described in example 4:

TABLE 8
Results

| | RQ value | | | |
|---|---|---|---|---|
| | 0.0002% | 0.0001% | | |
| | 3,5-di- | 3,5-di-caffeoyl- | 0.01% | |
| | caffeoyl- | quinic acid + | (+)- | Synergy |
| | quinic | 0.005% (+)- | alpha- | Index |
| Gene | acid | alpha-tocopherol | tocopherol | SI |
| HPRT1 | 1.0 | 1.0 | 1.0 | |
| (housekeeping) | | | | |
| CRYAB | 2.7 | 2.8 | 0.5 | 3.1 |
| DSG1 | 2.4 | 2.3 | 0.7 | 2.2 |

The results clearly show that surprisingly a combination of 3,5-di-caffeoylquinic acid and (+)-alpha-tocopherol relevantly and furthermore also synergistically up-regulates CRYAB gene. RQ value for the up-regulation of DSG1 was in this experiment slightly below 2.5, however, a synergistic effect was also observed.

(+)-Alpha-tocopherol alone when tested at 0.01% does not relevantly modulate any of the selected genes.

### Example 7

### Synergistic Effect of a Dicaffeoylquinic Acid Containing Plant Extract and Tocopherol on the Modulation of Gene Expression of Keratinocytes

Neonatal human epidermal keratinocytes (nHEK) were cultivated as described above and treated for 24 hours with yarrow extract of example 3 (Achillea millefolium, freeze-dried fresh press juice of flowering aerials, cultivated in Germany) and (±)-α-tocopherol (CAS number 10191-41-0, synonym: Synonym: DL-α-Tocopherol, Vitamin E, Sigma-Aldrich, synthetic) alone and in two combinations. Afterwards fast real-time qPCR was performed as described in above using a customised gene array.

For upregulations, RQ values ≥2.5 are considered to be relevant.

### Combination A:

The tested combination A (test concentration 0.005%) is composed of 50% yarrow extract (test concentration of 0.0025% in the combination) and 50% alpha-tocopherol (test concentration of 0.0025% in the combination).

Kull's equation was used for the calculation of the synergism index SI as described in example 4:

TABLE 9
Results combination A

| | RQ value | | | |
|---|---|---|---|---|
| | | Combination A: | | |
| | | 0.0025% | | |
| | 0.005% | Yarrow extract + | 0.005% | |
| | Yarrow | 0.0025% alpha- | alpha- | Synergy |
| Gene | extract | tocopherol | tocopherol | Index SI |
| HPRT1 | 1.0 | 1.0 | 1.0 | |
| (housekeeping) | | | | |
| HSPB8 | 4.6 | 4.0 | 1.2 | 2.1 |
| CRYAB | 14.0 | 11.3 | 1.3 | 4.8 |
| CLDN1 | 3.1 | 3.0 | 1.3 | 1.6 |
| OCLN | 2.8 | 3.6 | 2.4 | 1.4 |
| DEFB1 | 2.1 | 3.3 | 1.7 | 1.8 |
| DSC1 | 20.4 | 19.7 | 1.3 | 8.1 |
| DSG1 | 8.2 | 9.9 | 1.4 | 4.1 |

The results clearly show that surprisingly combination A of yarrow as a di-caffeoylquinic acid containing extract and alpha-tocopherol relevantly and furthermore also synergistically up-regulates HSPB8, CRYAB, CLDN1, OCLN, DEFB1, DSC1 and DSG1 genes.

Furthermore, calculating the synergy index SI for HSPB2 according to Kull's equation resulted in SI=1.6, which also represents a synergistic effect with regard to HSPB2.

Alpha-tocopherol alone when tested at 0.005% does not relevantly modulate any of the selected genes.

### Combination B:

The tested combination (test concentration 0.005%) is composed of 10% yarrow extract (test concentration of 0.0005% in the combination) and 90% alpha-tocopherol (test concentration of 0.0045% in the combination).

Kull's equation was also used for the calculation of the synergism index SI of combination B:

$$SI = CxD / A + CxE / B$$

With

A=RQ value by yarrow extract at concentration x

B=RQ value by tocopherol at concentration y

C=RQ value by the combination of yarrow extract at concentration x/10 and tocopherol at concentration 9y/10

D=Factor for yarrow extract=>0.1 (due to x/10 concentration tested in the combination)

E=Factor for tocopherol=>0.9 (due to 9y/10 concentration tested in the combination)

TABLE 10
Results combination B

| | RQ value | | | |
|---|---|---|---|---|
| | | Combination B: | | |
| | | 0.0005% | | |
| | 0.005% | Yarrow extract + | 0.005% | |
| | Yarrow | 0.0045% alpha- | alpha- | Synergy |
| Gene | extract | tocopherol | tocopherol | Index SI |
| HPRT1 | 1.0 | 1.0 | 1.0 | |
| (housekeeping) | | | | |
| HSPB8 | 2.6 | 3.1 | 2.3 | 1.3 |
| CLDN1 | 1.8 | 2.3 | 1.8 | 1.3 |
| DSC1 | 10.6 | 3.9 | 1.9 | 1.9 |
| DSG1 | 5.8 | 2.7 | 2.0 | 1.3 |

The results in the table clearly show that also combination B composed of yarrow as a dicaffeoylquinic acid containing extract and alpha-tocopherol relevantly and furthermore also synergistically up-regulates HSPB8, CLDN1, DSC1 and DSG1 genes.

Alpha-tocopherol alone when tested at 0.005% does not relevantly modulate any of the selected genes.

### Example 8

### Synergistic Effect of a Dicaffeoylquinic Acid Containing Plant Extract and Tocopherol on the Modulation of Gene Expression of Keratinocytes

Neonatal human epidermal keratinocytes (nHEK) were cultivated as described above and treated for 24 hours with yarrow extract of example 3 (Achillea millefolium, freeze-dried fresh press juice of flowering aerials, cultivated in Germany) and (+)-α-tocopherol (CAS number 59-02-9, synonym: Synonym: D-α-tocopherol, Vitamin E, Sigma-Aldrich) alone and in combination. Afterwards fast real-time qPCR was performed as described in above using a customised gene array.

For upregulations, RQ values ≥2.5 are considered to be relevant.

The tested combination (test concentration 0.00375%) is composed of 40% yarrow extract (test concentration of 0.0015% in the combination) and 60% (+)-alpha-tocopherol (test concentration of 0.00225% in the combination).

Kull's equation was used for the calculation of the synergism index SI:

$$SI = CxD / A + CxE / B$$

With

A=RQ value by yarrow extract at concentration x

B=RQ value by tocopherol at concentration y

C=RQ value by the combination of yarrow extract at concentration x/4 and tocopherol at 10 concentration 3y/4

D=Factor for yarrow extract=>0.25 (due to x/4 concentration tested in the combination)

E=Factor for tocopherol=>0.75 (due to 3y/4 concentration tested in the combination)

TABLE 11
Results

| | RQ value | | | |
|---|---|---|---|---|
| | | 0.0015% | | |
| | 0.006% | Yarrow extract + | 0.003% | |
| | Yarrow | 0.00225% (+)-alpha- | (+)-alpha- | Synergy |
| Gene | extract | tocopherol | tocopherol | Index SI |
| HPRT1 | 1.0 | 1.0 | 1.0 | |
| (housekeeping) | | | | |
| CRYAB | 7.3 | 5.0 | 0.7 | 5.5 |
| DEFB1 | 2.3 | 2.9 | 0.8 | 3.0 |
| DSC1 | 4.4 | 3.1 | 0.6 | 3.9 |
| DSG1 | 3.5 | 3.5 | 0.4 | 6.0 |

The results clearly show that surprisingly combination A of yarrow extract and (+)-alpha-tocopherol relevantly and furthermore also synergistically up-regulates CRYAB, DSC1 and DSG1 genes.

(+)-Alpha-tocopherol alone when tested at 0.003% does not relevantly modulate any of the selected genes.

### Example 9

### Stability Improvement of Caffeoylquinic Acids in Cosmetic Formulations

### Example 9.1

### Stability Tests

Chemical instability and formation/degradation to other compounds is a characteristic feature of caffeoylquinic acids. They are highly susceptible to the influence of temperature. As temperature increases they are not only more readily subjected to intramolecular isomerization and transesterification but also to degradation. To evaluate this aspect in a typical cosmetic formulation, an o/w emulsion containing 0.25% of yarrow extract with and without stabilizers according to table 12 was prepared. 3,4-, the sum of 1,5-/3,5- and 4,5-dicaffeoylquinic acids were determined by HPLC after preparation (start) and after 1, 3 and 6 months of storage at 40° C.

### Stabilizers:

DL-alpha Tocopherol (synonym: (±)-alpha Tocopherol), SymDecanox HA (Symrise), Oxynex ST Liquid (Merck) und EDTA BD (BASF)

TABLE 12
Formulas

| | | | w/w % | | | | |
|---|---|---|---|---|---|---|---|
| | Raw Material | INCI | A1 | A2 | A3 | A4 | A5 |
| A | Emulsiphos | Potassium Cetyl | 2.00 | 2.00 | 2.00 | 2.00 | 2.00 |
| | | Phosphate, Hydrogenated | | | | | |
| | | Palm Glycerides | | | | | |
| | Cutina PES | Pentaerythrityl | 1.50 | 1.50 | 1.50 | 1.50 | 1.50 |
| | | Distearate | | | | | |
| | Cutina GMS | Glyceryl Stearate | 1.50 | 1.50 | 1.50 | 1.50 | 1.50 |
| | SymMollient S | Cetearyl Nonanoate | 3.00 | 3.00 | 3.00 | 3.00 | 3.00 |
| | Vegetable Oil | Caprylic/Capric | 7.00 | 7.00 | 7.00 | 7.00 | 7.00 |
| | Triglyceride | Triglyceride | | | | | |
| | Dragoxat 89 | Ethylhexyl | 3.00 | 3.00 | 3.00 | 3.00 | 3.00 |
| | | Isononanoate | | | | | |
| B | Keltrol CG | Xanthan Gum | 0.15 | 0.15 | 0.15 | 0.15 | 0.15 |
| | Aristoflex AVC | Ammonium | 0.50 | 0.50 | 0.50 | 0.50 | 0.50 |
| | | Acryloyldimethyltaurate/ | | | | | |
| | | VP Copolymer | | | | | |
| C | Water dem. | Water (Aqua) | Ad 100 | | | | |
| | DL-alpha | Tocopherol | — | 0.50 | — | — | — |
| | Tocopherol | | | | | | |
| | SymDecanox HA | Caprylic/Capric | — | — | 2.00 | — | — |
| | | Triglyceride, | | | | | |
| | | Hydroxymethoxyphenyl | | | | | |
| | | Decanone | | | | | |
| | Oxynex ST Liquid | Diethylhexyl | — | — | — | 0.50 | — |
| | | Syringylidenemalonate, | | | | | |
| | | Caprylic/Capric | | | | | |
| | | Triglyceride | | | | | |
| | EDTA BD | Disodium EDTA | — | — | — | — | 0.10 |
| | Hydrolite-5 | Pentylene Glycol | 3.00 | 3.00 | 3.00 | 3.00 | 3.00 |
| | SymSave H | Hydroxyacetophenone | 0.60 | 0.60 | 0.60 | 0.60 | 0.60 |
| | Propylene Glycol | Propylene Glycol | 3.00 | 3.00 | 3.00 | 3.00 | 3.00 |
| | Glycerin 85% | Glycerin | 1.00 | 1.00 | 1.00 | 1.00 | 1.00 |
| D | Sodium Hydroxide, | Sodium Hydroxide | As required for pH adjustment to 5.0 | | | | |
| | 10% sol. | | | | | | |
| | Citric acid 30% | Citric Acid | | | | | |
| | sol. | | | | | | |
| E | Yarrow extract* | | 0.25 | 0.25 | 0.25 | 0.25 | 0.25 |
| | SUM | | 100.0 | 100.0 | 100.0 | 100.0 | 100.0 |

| | |
|---|---|
| PH value | pH 5.0 |

*Yarrow extract as described in example 3

TABLE 13
Storage

| Duration of | A1 (no stabilizer) | | | | A2 (0.5% DL-alpha Tocopherol) | | | |
|---|---|---|---|---|---|---|---|---|
| storage at 40° C. | 3,4- | 3,5-* | 4,5- | Sum | 3,4- | 3,5-* | 4,5- | Sum |
| Start | 30 | 25 | 31 | 86 | 34 | 29 | 35 | 98 |
| 1 months | 28 | 23 | 27 | 58 | 34 | 27 | 33 | 94 |
| 3 months | 15 | 11 | 13 | 39 | 31 | 23 | 28 | 82 |
| Reduction vs | | | | 55% | | | | 16% |
| Start [%] | | | | | | | | |
| 6 months | 8 | 4 | 7 | 19 | 27 | 18 | 24 | 69 |
| Reduction vs | | | | 78% | | | | 29% |
| Start [%] | | | | | | | | |

| Duration of | A3 (2% SymDecanox HA) | | | | A4 (0.5% Oxynex ST Liquid) | | | |
|---|---|---|---|---|---|---|---|---|
| storage at 40° C. | 3,4- | 3,5-* | 4,5- | Sum | 3,4- | 3,5-* | 4,5- | Sum |
| Start | 33 | 28 | 34 | 95 | 33 | 28 | 34 | 95 |
| 1 months | 30 | 25 | 30 | 85 | 33 | 28 | 33 | 94 |
| 3 months | 17 | 13 | 16 | 46 | 19 | 16 | 18 | 53 |
| Reduction vs | | | | 51% | | | | 44% |
| Start [%] | | | | | | | | |
| 6 months | 8 | 5 | 9 | 22 | 11 | 9 | 11 | 31 |
| Reduction vs | | | | 77% | | | | 67% |
| Start [%] | | | | | | | | |

| Duration of | A5 (0.1% EDTA BD) | | | |
|---|---|---|---|---|
| storage at 40° C. | 3,4- | 3,5-* | 4,5- | Sum |
| Start | 34 | 29 | 35 | 98 |
| 1 months | 33 | 28 | 33 | 94 |
| 3 months | 20 | 17 | 18 | 55 |
| Reduction vs | | | | 44% |
| Start [%] | | | | |
| 6 months | 11 | 9 | 11 | 31 |
| Reduction vs | | | | 68% |
| Start [%] | | | | |

*1,5- and 3,5-Dicaffeoyl-quinic acid exhibited overlaying peaks and were not sufficiently separated by the used HPLC method and are therefore given as sum.

The results clearly show that without addition of a stabilizer the sum of 3,4-, 1,5-/3,5- and 5 4,5-dicaffeoylquinic acids decrease by 55% after 3 months and by 78% after 6 months when stored at 40° C.

Addition of 1% of SymDecanox HA (A3) exhibits almost no stabilizing effect on the sum of 3,4-, 1,5-/3,5- and 4,5-dicaffeoylquinic acids versus A1 with decrease by 51% after 3 months and by 77% after 6 months when stored at 40° C.

Addition of 0.5% Oxynex ST Liquid (A4) and 0.1% EDTA BD (A5) only exhibit a slight stabilizing effect on the sum of 3,4-, 1,5-/3,5- and 4,5-dicaffeoylquinic acids versus A1 with decrease by 44% after 3 months and by 67 and 68% after 6 months when stored at 40° C.

Surprisingly, the most prominent and best stabilization is obtained by addition of 0.5% alpha tocopherol (A2). The sum of 3,4-, 1,5-/3,5- and 4,5-dicaffeoylquinic acids versus A1 is decreased only by 16% after 3 months and only by 29% after 6 months when stored at 40° C.

Combinations of 3,4-, 1,5-/3,5- and 4,5-dicaffeoylquinic acid with tocopherol therefore not only show synergistic activity as shown in examples 4, 5 and 6, but are also best preserved by this compound in water containing formulas such as typical cosmetic formulations.

### Example 9.2

### Discoloration Tests

Additionally to the determination of dicaffeoylquinic acid content, samples from storage at 40° C. were also evaluated for discoloration. Color can be described using the CIELAB color model, which is based on an opponent color system. CIELAB indicates the color by values on three axes: L*, at, and b+with dimension L for lightness and a* and b* for the color-opponent dimensions red/green and yellow/blue, based on nonlinearly compressed coordinates. The L* axis extends from black (0) to white (100), the a* axis from green (−a) to red (+a) and the b* axis from blue (−b) to yellow (+b).

Chromametry measurements of samples at baseline and after storage for 6 months at 40° C. were performed using a Chroma Meter CR 410 (Konica/Minolta). The difference of 2 colors ΔE can be calculated using the following equation:

$$Δ {E}_{p , v} = \sqrt{{( {L}_{p}^{*} - {L}_{v}^{*} )}^{2} + {( {a}_{p}^{*} - {a}_{v}^{*} )}^{2} + {( {b}_{p}^{*} - {b}_{v}^{*} )}^{2}}$$

with p=sample 1 and v=sample 2.

The results are shown in Table 14.

TABLE 14
Results

| | | A2 (0.5% DL- | A4 (0.5% | |
|---|---|---|---|---|
| | A1 (no | alpha | Oxynex ST | A5 (0.1% |
| | stabilizer) | Tocopherol | Liquid) | EDTA) |
| L* value | | | | |
| Start | 86.17 | 86.64 | 86.62 | 86.30 |
| 6 months | 79.51 | 83.93 | 80.99 | 78.49 |
| a* value | | | | |
| Start | 0.38 | 0.34 | 0.18 | 0.36 |
| 6 months | 3.27 | 1.19 | 2.70 | 3.31 |
| b* value | | | | |
| Start | 8.97 | 8.96 | 8.95 | 9.30 |
| 6 months | 12.77 | 12.79 | 12.28 | 13.12 |
| ΔE | 8.51 | 4.77 | 7.01 | 9.18 |

The results clearly show that also discoloration is lowest for the formulation with alpha tocopherol (ΔE 4.77 for A2 versus 8.51 for A1).

A slight improvement was observed by addition of Oxynex ST liquid (ΔE 7.01 for A4 versus 8.51 for A1). Addition of EDTA even resulted in a slightly higher color change than the formulation without stabilizer (ΔE 9.18 for A5 versus 8.51 for A1).

### Example 10

### Modulation of the CRYAB Protein Level In Ex Vivo

For evaluation of the ex vivo skin HSP increasing efficacy of dicaffeoylquinic acids in combination with tocopherol, a yarrow extract containing cosmetic formulation was used:

The used yarrow extract was composed of 25% of yarrow press juice dry matter and 75% maltodextrin DE 17-20 and was prepared by spray-drying. It contained 0.26% 3,4-dicaffeoylquinic acid, 0.20% 1,5- and 3,5-dicaffeoylquinic acid in sum and 0.23% of 4,5-dicaffeoylquinic acid (0.69% dicaffeoylquinic acids in sum).

The used DL-alpha-tocopherol (BASF) was characterized by a purity of 97% (GC) and an optical rotation of 0.00°. The following cosmetic formulations were prepared:

TABLE 15
Cosmetic formulations

| | | Amount [w/w %] | | | |
|---|---|---|---|---|---|
| | INCI | 10.1 | 10.2 | 10.3 | 10.4 |
| Phase A | | | | | |
| PCL-Liquid 100 | Cetearyl | 3.0 | 3.0 | 3.0 | 3.0 |
| | Ethylhexanoate | | | | |
| Lanette O | Cetearyl Alcohol | 2.0 | 2.0 | 2.0 | 2.0 |
| Mineral oil 5° E | Mineral oil | 3.0 | 3.0 | 3.0 | 3.0 |
| Eutanol G | Octyldodecanol | 4.0 | 4.0 | 4.0 | 4.0 |
| Abil 350 | Dimethicone | 0.5 | 0.5 | 0.5 | 0.5 |
| Phase B | | | | | |
| Pemulen TR-1 | Acrylates/C10-30 | 0.2 | 0.2 | 0.2 | 0.2 |
| | Alkyl Acrylate | | | | |
| | Crosspolymer | | | | |
| Carbopol Ultrez- | Acrylates/C10-30 | 0.05 | 0.05 | 0.05 | 0.05 |
| 21 | Alkyl Acrylate | | | | |
| | Crosspolymer | | | | |
| Water | | Ad 100 | Ad 100 | Ad 100 | Ad 100 |
| Hydrolite ® 5 | Pentyleneglycol | 2.0 | 2.0 | 2.0 | 2.0 |
| Phase C | | | | | |
| Sodium | Aqua, Sodium | 0.5 | 0.5 | 0.5 | 0.5 |
| Hydroxide 10% | Hydroxide | | | | |
| solution | | | | | |
| Phase D | | | | | |
| Yarrow extract | Maltodextrin, Achillea | — | 0.025 | 0.025 | — |
| | Millefolium Extract | | | | |
| DL-Alpha- | Tocopherol | — | — | 0.05 | 0.05 |
| Tocopherol | | | | | |
| Sum | | 100.0 | 100.0 | 100.0 | 100.0 |

### Production Method:

Heat phase B at 80° C. without Pemulen TR-1. Disperse Pemulen TR-1 by stirring. Heat phase A at 80° C. and add A to B with an Ultra Turrax. Allow to cool down at 40° C. Adjust the pH value with phase C at 5.5.
Add Phase D to obtain formulation 10.2 to 10.4.

Organ culture of human skin was performed starting from a skin sample, exciding pieces of approximately 8×3 mm (∅×thickness) and culturing them up to day 6. Skin samples (6 per treatment) were cultured in an air-liquid interface in a perforated ring of stainless steel in contact with culture medium (modified Williams' E medium). The culture medium was renewed at the day 3.

Formulations 10.1 to 10.4 were applied topically and renewed daily. For application the skin biopsies were gently cleaned with a cotton pad and then 4 μl of each formulation were applied on top of each piece and covered with a 6 ∅ mm delivery membrane.

After 6 days of organ culture, histological sections were prepared from the skin samples and 12 skin sections were immunostained with the selected antibody (CRYAB: Santa Cruz Biotechnology cat#sc-137129). The amount of antigen present in each slide were evaluated by determining the intensity and the distribution of the pink/red within the epidermis using Image J (NIH-USA). The obtained data were then normalized upon the dimension of the analyzed surface expressed in pixels.

Results are summarized in table 16.

TABLE 16

| | Mean | Modulation | Statistical |
|---|---|---|---|
| | CRYAB | in % vs 10.1 | significance |
| Test sample | score | (placebo) | p (t test) vs 10.1 |
| Untreated | 32.48 | — | — |
| Formulation 10.1 | 31.29 | — | — |
| (placebo) | | | |
| Formulation 10.2 (0.025% | 34.20 | +9.3% | p > 0.05 |
| yarrow extract) | | | |
| Formulation 10.3 (0.025% | 36.67 | +17.2% | p < 0.01 |
| yarrow extract + 0.05% | | | |
| tocopherol) | | | |
| Formulation 10.4 (0.05% | 31.13 | −0.5% | p > 0.05 |
| tocopherol) | | | |

The results clearly show that the placebo (formulation 10.1) had no relevant effect on the mean CRYAB score when compared to untreated.

0.05% tocopherol alone (formulation 10.4) had no relevant effect on the mean CRYAB score when compared to placebo.

0.025% yarrow extract (corresponding to 0.00017% dicaffeoylquinic acids in sum) alone (formulation 10.2) led to a slight increase of 9.3% of the mean CRYAB score, however, which was calculated as not being significant versus placebo.

The combination of 0.05% tocopherol and 0.00017% dicaffeoylquinic acids contained in 0.025% yarrow extract (formulation 10.3) led to a significant (p<0.01) modulation of the mean CRYAB score by 17.2% versus placebo. This effect is clearly synergistic as the expected additive modulation versus placebo taking into account the modulation achieved by formulation 10.2 and 10.4 would only have been +8.8%.

Compared to organ culture of human skin (ex vivo), the concentration of the combination of tocopherol and dicaffeoylquinic acids, which is effectively applied on the skin, and with this the concentration of the yarrow extract, which is effectively applied on the skin, is typically 2 to 5 fold higher when topically applied on in vivo human skin. This is due to the different conditions in organ culture, which lead to lower effectively applied concentrations.

The used gel formulation allows very good liberation of the active ingredients after topical application. Other typically used cosmetic formulations like e.g. o/w emulsions depending on their composition will allow a less readily liberation which will lead to a typically 4 to 20 fold higher required use level of the dicaffeoylquinic acids, and with this required use level of the dicaffeoylquinic acids extracts to exhibit the desired effect.

### Example 11

### Formulation Examples

1=Well ageing Face Cream, soft touch
2=Daily emulsion with SPF 30, UVA/UVB balanced
3=Protecting and scalp soothing hair conditioner, rinse off
4=Barrier strengthening oil in cream shower gel
5=Fresh hair shampoo
6=Well-ageing eye contour serum
7=Natural beauty enhancer (solid concentrate)
8=Refreshing gel with UV protection
9=Hand and body cream
10=Natural beauty protecting soft wet wipes
11=Weather Protection cream
12=Men After Shave fluid
13=Reinforcing purifying mask, rinse-off
14=Beach time lotion SFP 30
15=Skin well-being body lotion
16=Refreshing Eau micellaire
17=Reinforcing Sun Protection Mist exp. SPF30, UVA/UVB Balance

In formulations 1 to 17 the following perfume oils PF01 and PF02 were each used as fragrance (DPG=dipropylene glycol).

TABLE 17
Composition of perfume oil 1 (PO1, Amounts in ‰ b.w.)

| Ingredients | Amount |
|---|---|
| ALDEHYDE C14 SO-CALLED | 2 |
| ALLYL AMYL GLYCOLATE 10% DPG | 5 |
| ANISIC ALDEHYDE PURE | 5 |
| APPLE OLIFFAC TYPE | 10 |
| BENZYLACETATE | 50 |
| BERGAMOT IDENTOIL ® COLOURLESS | 15 |
| CANTHOXAL | 5 |
| CETALOX 10% IPM | 3 |
| CITRONELLOL 950 | 40 |
| DAMASCENONE TOTAL 1% DPG | 5 |
| DAMASCONE ALPHA 10% DPG | 5 |
| DAMASCONE DELTA 10% DPG | 2 |
| DIMETHYL BENZYL CARBINYL BUTYRATE | 2 |
| DIPROPYLENE GLYCOL | 178 |
| EBANOL | 2 |
| ETHYL DECADIENOATE TRANS CIS-2,4 10% IPM | 2 |
| FLOROSA | 5 |
| FRAMBINON ® 10% DPG | 7 |
| GALAXOLIDE 50% IN IPM | 100 |
| GALBEX TYPE BASE | 1 |
| GERANYL ACETATE PURE | 2 |
| HEDIONE | 30 |
| HELIOTROPIN | 10 |
| HEXENYL ACETATE CIS-3 10% DPG | 1 |
| HEXENYL SALICYLATE CIS-3 | 5 |
| HEXYL CINNAMIC ALDEHYDE ALPHA | 70 |
| HEXYL SALICYLATE | 50 |
| HYDROXY CITRONELLAL | 10 |
| ISO E SUPER | 15 |
| ISORALDEINE 70 | 20 |
| LEAFOVERT ® | 1 |
| LILIAL | 60 |
| LINALOOL | 60 |
| LINALYL ACETATE | 20 |
| LYRAL | 7 |
| MANZANATE | 2 |
| PHENOXANOL | 7 |
| PHENYLETHYL ALCOHOL | 120 |
| SANDAL MYSORE CORE | 2 |
| SANDRANOL ® | 7 |
| STYRALYL ACETATE | 3 |
| TAGETES RCO 10% TEC | 2 |
| TERPINEOL PURE | 20 |
| TETRAHYDROGERANIOL 10% DPG | 5 |
| TONALIDE | 7 |
| VERTOCITRAL 10% DPG | 5 |
| VERTOFIX | 15 |
| Total | 1000 |

TABLE 18
Composition of perfume oil 2 (PO2, Amounts in ‰ b.w.)

| Ingredients | Amount |
|---|---|
| AMBRETTOLIDE (MACRO) | 10 |
| AMBROXIDE 10% in IPM | 10 |
| BENZYL ACETATE | 20 |
| BENZYL SALICYLATE | 15 |
| BERGAMOT OIL. bergapten-free | 60 |
| CALONE ® 1951 10% in DPG | 15 |
| COUMARIN | 5 |
| CYCLOGALBANATE ® 10% in DPG | 10 |
| ALPHA -DAMASCONE 1% in DPG | 20 |
| DIHYDROMYRCENOL | 10 |
| ETHYL LINALOOL | 75 |
| ETHYL LINALYLACETATE | 50 |
| ETHYL MALTOL 1% in DEP | 10 |
| ETHYLENE BRASSYLATE (MACRO) | 80 |
| FLOROSA | 40 |
| GERANYLACETATE | 10 |
| HEDIONE ® HC/30 | 35 |
| HEDIONE ® | 210 |
| HELIONAL ® | 15 |
| HELVETOLIDE ® (ALICYC) | 30 |
| HEXENYLSALICYLATE CIS-3 | 20 |
| ISO E SUPER ® | 40 |
| LEAFOVERT ® 10% in DEP | 10 |
| LILIAL ® | 80 |
| LYRAL ® | 20 |
| MANDARIN OIL | 10 |
| STYRALYL ACETATE | 5 |
| SYMROSE ® | 15 |
| VANILLIN 10% in DEP | 20 |
| DIPROPYLENE GLYCOL (DPG) | 50 |
| TOTAL | 1000 |

TABLE 19
Cosmetic formulations 1 to 11 (amounts in % b.w.)

| | Amounts [wt.-%] | | | | | | | | | | |
|---|---|---|---|---|---|---|---|---|---|---|---|
| Ingredients INCI name | 1 | 2 | 3 | 4 | 5 | 6 | 7 | 8 | 9 | 10 | 11 |
| 3,4-Dicaffeoylquinic acid | 0.002 | | | | | | | | | | |
| 3,5-Dicaffeoylquinic acid | | | | | 0.005 | | | | | | |
| 4,5-Dicaffeoylquinic acid | | | | 0.01 | 0.002 | | | | | | |
| Yarrow Extract Powder (25% extract dry matter) Maltodextrin, | | 0.3 | | | | | 0.25 | | | | 0.1 |
| Achillea Millefolium Extract | | | | | | | | | | | |
| Green coffee bean extract powder (70% extract dry matter) | | | 0.1 | | | 0.1 | | | 0.05 | | |
| Coffea robusta seed extract, Maltodextrin | | | | | | | | | | | |
| Chrysanthemum Flower Extract Powder Chrysanthemum indica | | | | | | 0.1 | | | | 0.06 | |
| Extract | | | | | | | | | | | |
| Honeysuckle Flower Extract Powder Lonicera Japonica Extract | | | | | | | | 0.2 | | | 0.1 |
| Tocopherol | 0.2 | 0.4 | 0.15 | 0.3 | 0.05 | 0.2 | 0.5 | 0.5 | 0.1 | 0.3 | 0.2 |
| Tocopherol | | | | | | | | | | | |
| Actipone Alpha-Pulp Aqua, Butylene Glycol, Malic acid, Actinidia | | | | | 0.2 | | | | | | |
| chinensis fruit extract, citrus aurantium dulcis juice, citrus paradise | | | | | | | | | | | |
| juice, pyrus malus juice | | | | | | | | | | | |
| Acusol OP 301 Styrene/Acrylates Copolymer | | | | 1.0 | | | | | | | |
| Aloe Vera Gel Concentrate 10/1 Aloe Barbadensis Leaf Juice | | | | | | | | 0.3 | | | |
| Amigum Sclerotium Gum | | | | | | 0.2 | | | | | |
| Antil 120 Plus PEG-120 Methyl Glucose Dioleate | | | | 1.0 | | | | | | | |
| Apricot Kernel Oil Prunus Armeniaca Kernel Oil | | | 0.5 | | | | | | | | |
| Argan Oil Argania Spinosa Kernel Oil | | | 0.5 | | | | | | | | |
| Aristoflex ® AVC Ammonium acryloyoldimethyltaurate/vp | | | | | | | | 1.4 | | | |
| copolymer | | | | | | | | | | | |
| Beeswax White Cera Alba | | | | | | | 14.0 | | | | 2.0 |
| Benzyl Alcohol Benzyl Alcohol | | | | | | | 0.5 | | | | |
| Biotive L-Arginine Arginine | | 1.5 | | | | | | 1.0 | | | |
| Caffeine Caffeine | | | | | | 0.1 | | | | | |
| Carnauba Wax LC organic Copernicia Cerifera (Carnuba) Wax | | | | | | | 6.0 | | | | |
| Carbopol ETD 2050 Polymer Carbomer | | 0.3 | | | | | | | | | |
| Carbopol Ultrez 21 Acrylates/c10-30 Alkyl Acrylate Crosspolymer | | | | | | | | | 0.2 | | |
| Cetiol Sensoft Propylheptyl caprylate | | 4.0 | | | | | | | | | |
| Citric acid 10% solution Aqua, Citric acid | | | | 0.5 | | | | | | 0.1 | |
| Cocoa Butter Theobroma Cacao (Cocoa) Seed Butter | | | | | | | 3.0 | | | | 1.0 |
| Coconut oil Cocos Nucifera (Coconut) Oil | | | | | | | 1.0 | | | | |
| Colour Colour | | | | | 0.35 | | | | | | |
| Comperlan ® IP Cocamide MIPA | | | | 1.0 | | | | | | | |
| Crinipan AD Climbazole | | | | | 0.2 | | | | | | |
| Crinipan PMC green Propanediol caprylate | | | 0.5 | | | | | | | | |
| Cutina GMS V Glyceryl Stearate | | | | | | | | | 1.0 | | |
| Cutina HR Powder Hydrogenated Castor Oil | | | | | | | | | | | 1.0 |
| Cyclomethicone Cyclomethicone | | | | | | | | | | 0.2 | |
| Dehyton PK 45 Cocamidopropyl Betaine | | | | 8.0 | 8.0 | | | | | | |
| Dehyquart A CA Cetrimonium Chloride | | | 0.5 | | | | | | | | |
| Dehyquart SP Quaternium-52 | | | 4.0 | | | | | | | | |
| Dermofeel GO Soft Polyglyceryl-2 Sesquioleate | | | | | | | | | | | 2.5 |
| Dermofeel sensolv/soamyl Laurate | | | | | | | | | | | 7.0 |
| Dermosoft 1388 eco Glycerin, Aqua, Sodium Levulinate, Sodium | | | | | | | | | | | 3.0 |
| Anisate | | | | | | | | | | | |
| Dimethicone Dimethicone | | | 0.1 | | | | | | | | |
| Dracorin CE Glyceryl Stearate Citrate | 2.0 | | 1.0 | | | 3.0 | | | | | |
| Dracorin GOC Glyceryl Oleate Citrate, Caprylic/Capric Triglyceride | | | | | | 1.5 | | | 2.0 | | |
| DragoCalm Aqua, Glycerin, Avena sativa kernel extract | | | | | | | | | 1.5 | | |
| Dragosantol 100 Bisabolol | | | | | | | | | | 0.1 | |
| Dragosine Carnosine | 0.1 | | | | | | | 0.2 | | | |
| Dragoxat 89 Ethylhexyl Isononanoate | 2.0 | | | | | 3.0 | | | 3.0 | | |
| EDTA BD Disodium EDTA | | 0.1 | 0.1 | 0.1 | 0.1 | 0.1 | | 0.1 | | | |
| Emulgin B2 Ceteareth-20 | | | | | | | | | | 2.5 | |
| Emulsiphos Potassium Cetyl Phosphate, Hydrogenated Palm | | 2.5 | | | | | | | | | |
| Glycerides | | | | | | | | | | | |
| Estearina L2SM GS Stearic Acid, Palmitic Acid | | | | 0.5 | | | | | | | |
| Ethanol Ethanol | | | | | | | | | | | 5.0 |
| Euxyl K300 Methyl-, Butyl-, Ethyl-, Propyl, Isobutylparaben, | | | | | | | | | 0.3 | | |
| Phenoxyethanol | | | | | | | | | | | |
| Floraesters K-100 ® Jojoba Hydrolyzed jojoba esters, Jojoba | | | | | | | | 1.0 | | | |
| esters, Aqua | | | | | | | | | | | |
| Extrapone Arnica Flower Aqua, Arnica Montana Flower Extract, | | | | | | | | 2.0 | | 5.0 | |
| Propylene Glycol | | | | | | | | | | | |
| Extrapone Elderflower Aqua, Sambucus Nigra Flower Extract, | | | | 3.0 | | | | | | | |
| Propylene Glycol | | | | | | | | | | | |
| Extrapone Ivy Aqua, Sambucus Nigra Flower Extract, Propylene | | | | 5.0 | | | | | | | |
| Glycol | | | | | | | | | | | |
| Fragrance Parfum | 0.3 | 1.0 | 0.5 | 1.5 | 1.0 | 0.2 | 0.5 | 0.4 | 0.2 | 0.1 | 0.35 |
| Frescolat ML cryst Menthyl Lactate | | 0.5 | | | 1.0 | | | | | | |
| Frescolat Plus Menthol, Menthyl Lactate | | | 0.3 | | | | | | | | |
| Frescolat X-Cool Menthyl Ethylamido Oxalate | | | | | | | | 0.8 | | | |
| Glucotain Care Cocoyl Methyl Glucamide | | | | 2.0 | | | | | | | |
| Glycerin Glycerin | 2.0 | 1.0 | | | | 4.0 | | | 2.5 | 3.0 | 7.0 |
| Hemp Oil Cannabis Sativa Seed Oil | 1.0 | | | | | | | | | | |
| Hydrolite 5 Pentylene Glycol | | | | | | | 2.0 | | | 3.0 | |
| Hydrolite 6 1,2-Hexanediol | | | | | 1.0 | | | 0.5 | | | |
| Hydrolite 7 green 1,2-Heptanediol | | 1.0 | | | 1.0 | | | 2.0 | | | |
| Hydrolite CG Caprylyl Glycol | | | | | | | 0.5 | | | 1.0 | 0.3 |
| Hydroviton ® PLUS 2290 Aqua, Pentylene glycol, Glycerin, | | | | | | | | | | 1.5 | |
| Fructose, Urea, Citric acid, Sodium hydroxide, Maltose, Sodium pca, | | | | | | | | | | | |
| Sodium chloride, Sodium lactate, Trehalose, Allantoin, Sodium | | | | | | | | | | | |
| hyaluronate Glucose | | | | | | | | | | | |
| ImerCare ® Pharma 00T Talc | | | | | | | 2.0 | | | | |
| Isoadipate Diisopropyl Adipate | | 7.5 | | | | | | | | | |
| Isodragol Triisononanoin | | | | | | | | | 4.0 | | |
| Isopropyl Myristate Isopropyl myristate | | | | | | | | | | 0.5 | |
| Isopropyl Palmitate/sopropyl Palmitate | | | | | | | | | | 3.0 | |
| Jojoba Oil Simmondsia Chinensis Seed Oil | | | | | | | | | | 0.5 | |
| Lanette E Sodium cetearyl sulfate | | 0.8 | | | | | | | | | |
| Lanette O Cetearyl Alcohol | 5.0 | 1.5 | | 2.0 | | | | | 1.5 | 2.5 | |
| Lara Care A-200 Galactoarabinan | | | 0.5 | | | | | | | | |
| Magnesium Stearate Magnesium Stearate | | | | | | | | | | | 0.7 |
| Mango Butter Mangifera indica Seed Butter | | | | | | | Ad | | | | |
| | | | | | | | 100 | | | | |
| Mineral oil Paraffinum Liquidum | | | | | | | | | | 4.0 | |
| Nativacare 5600 Zea Mays (Corn Starch) | | | | | 2.0 | 1.5 | | | | | |
| Neo-Heliopan 303 Octocrylene | | | | | | | | 10.0 | | | |
| Neo-Heliopan 357 Butyl Methoxydibenzoylmethane | | 4.0 | | | | | | 3.0 | | | |
| Neo-Heliopan BMT Bis-Ethylhexyloxyphenol Methoxypenyl | | 3.0 | | | | | | 1.5 | | | |
| Triazine | | | | | | | | | | | |
| Neo-Heliopan Hydro Phenylbenzimidazole sulfonic acid | | 3.0 | | | | | | 2.0 | | | |
| Neo-Heliopan HMS Homosalate | | | | | | | | 2.5 | | | |
| Neo-Heliopan OS Ethylhexyl Salicylate | | 5.0 | | | | | | 5.0 | | | |
| Panthenol Panthenol | | | | | | | | | 0.5 | | |
| Passion Fruit oil Passiflora Edulis Seed Oil | 1.0 | | | | | | | | | | |
| PCL Liquid Cetearyl Ethylhexanoate, Isopropyl Myristate | | | | | | | | | 7.0 | | |
| PCL Liquid 100 Cetearyl Ethylhexanoate | | | 0.3 | | | 2.0 | | | | | |
| PCL Solid Stearyl Heptanoate, Stearyl Caprylate | | | 3.0 | | | | | | 2.5 | | |
| Phenoxyethanol Phenoxyethanol | | 0.5 | | | | | | | | | |
| Phytoconcentrole Carrot Helianthus Annuus Seed Oil, Beta- | | | | | | | | 0.3 | | | |
| Carotene, Daucus Carota Sativa Root Extract | | | | | | | | | | | |
| Phytoconcentrole Hemp CBD Cannabis Sativa Seed Oil, | | | | | | | 0.5 | | | | |
| Cannabidiol, Tocopherol | | | | | | | | | | | |
| Plantacare 2000 UP decyl Glucoside | | | | | 4.0 | | | | | | |
| Potassium Sorbate Potassium Sorbate | | | | | | | | | 0.1 | | |
| Propylene Glycol Propylene Glycol | | 3.0 | | | 2.0 | | | | | | |
| Sensiva SC 50 Ethylhexylglycerin | | | | | | | | | | 0.3 | |
| RetinoPeptide 189 Glycerin, Pentylene Glycol, Water, Myristoyl | 1.0 | | | | | | | | | | |
| Nonapeptide-3 | | | | | | | | | | | |
| Retinyl Palmitate Retinyl Palmitate | | | | | | 0.1 | | | | | |
| Rice Bran Wax Oryza Sativa (Rice) Bran Wax | | | | | | | 6.0 | | | | |
| Sensocel 10 Cellulose | | | | | | | | 3.0 | | | |
| Shea Butter (Organic) Butyrospermum Parkii Butter | | 0.3 | | | | | | | | | 1.0 |
| Sodium Hydroxide 10% solution Aqua, Sodium Hydroxide | | 2.0 | | | 2.2 | | | 0.2 | 0.5 | | |
| Softigen Pura Olus Oil | | | | | | | | | | | 5.0 |
| Squalane Squalane | | | | | | | | | | | 10.0 |
| Super Hartolan Lanolin Alcohol | | | | | | | | | | 0.2 | |
| Sweet Almond Oil Prunus Amygdalus Dulcis (Sweet Almond) oil | | | | | | | 23.5 | | | | |
| SymBright ® 2036 Sclareolide | 0.2 | | | | | | | | | | |
| SymBronze Caprylic/Capric Triglyceride, Isochrysis Galbana | | | | 1.0 | | | 0.7 | | | | |
| Extract | | | | | | | | | | | |
| SymCalmin Butylene Glycol, Pentylene Glycol, Hydroxyphenyl | | | 0.3 | | | | | | | | |
| Propamidobenzoic Acid | | | | | | | | | | | |
| SymCare O Hexyldecanol, Pentylene Glycol, 4-t-Butylcylohexanol, | | | | | | | | 2.0 | | | |
| Bisabolol, Cetylhydroxyproline Palmitamide, Hydroxyphenyl | | | | | | | | | | | |
| Propamidobenzoic acid, Stearic acid, Brassica campestris | | | | | | | | | | | |
| (Rapeseed)Sterols, Zingiber officinale (Ginger)Root extract | | | | | | | | | | | |
| SymCare W2 PEG-40 Hydrogenated Castor Oil, Trideceth-9, | | | | | | | | | | 1.5 | |
| Pentylene Glycol, 4-t-Butylcyclohexa-nol, Propylene Glycol, Water, | | | | | | | | | | | |
| Hydroxyphenyl Propamidobenzoic Acid | | | | | | | | | | | |
| SymClariol Decylene Glycol | | | | | 0.5 | | | | | | |
| SymControl Care Aqua, Glycerin, Tetraselmis Suecica Extract | | | | | | | | | | 1.0 | |
| SymControl Scalp Aqua, Glycerin, Mannitol, Tetraselmis Suecica | | | 0.7 | | | | | | | | |
| Extract | | | | | | | | | | | |
| SymDecanox HA Caprylic/Capric Triglyceride, | | 1.0 | | | | | 1.0 | | | | 0.5 |
| Hydroxymethoxyphenyl Decanone | | | | | | | | | | | |
| SymDetox 1711 3,3,5- Trimethylcyclohexyl Succinate | | | | | | 0.5 | | | | | |
| Dimethylamide | | | | | | | | | | | |
| SymDeo B125 2-methyl 5-cyclohexylpentanol | | | | | | | | 0.5 | | | |
| SymDeo Plus Lauryl alcohol, Phenoxyethanol, 2-benzylheptanol, | | | | | | | | 0.3 | | | |
| Decylene glycol | | | | | | | | | | | |
| SymDiol 68 1,2-Hexanediol, Caprylyl glycol | | | 0.5 | | | 0.5 | | | | | |
| SymDStress 100 Bisabolol, Cannabidiol | | | | | | | | 0.3 | | | |
| SymEffect Sun Cera Alba. Sodium Stearoyl Lactylate | | | | | | | | 1.5 | | | |
| SymFinity 1298 Echinacea Purpurea Extract | 0.1 | | | | | | | | | | |
| SymGlucan Aqua, Glycerin, 1,2-Hexanediol, Caprylyl Glycol, Beta- | 1.0 | | | | | | | | | 2.0 | |
| Glucan | | | | | | | | | | | |
| SymGuard CD Phenylpropanol, O-cymen-5-ol, Decylene glycol | | | | | | | | 0.5 | | | |
| SymHair Force 1631 Pentylene Glycol, Isochrysis Galbana Extract | | | | | 1.0 | | | | | | |
| SymHair Restore Glycerin, Triticum vulgare protein, Aqua | | | 1.0 | | | | | | | | |
| SymHair Shield Pentylene Glycol, Aqua, Glycerin, Triticum vulgare | | | | | 1.0 | | | | | | |
| bran extract, 1,2-Hexanediol, Capryly glycol | | | | | | | | | | | |
| SymLift Aqua, Trehalose, Glycerin, Pentylene Glycol, Beta-Glucan, | 1.5 | | | | | 5.0 | | | | | |
| Hordeum vulgare seed extract, sodium hyluronate, 1,2-Hexanediol, | | | | | | | | | | | |
| Caprylyl Glycol | | | | | | | | | | | |
| SymLite G8 Glyceryl Caprylate | 0.3 | | 0.2 | | | | 0.5 | | 0.2 | | 0.5 |
| SymMatrix Maltodextrin, Rubus Fruticosus Leaf Extract | | 0.2 | | | | | | | | | |
| SymMollient PDCC Propanediol Dicaprylate/Caprate | 3.0 | | | | | | | 2.0 | | | |
| SymMollient S green Cetearyl Nonanoate | 2.0 | 1.0 | | 3.0 | | 2.0 | | | | | |
| SymMollient W/S Trideceth-9, PEG-5 Isononanoate, Aqua | | | | 3.0 | | | | | | | |
| SymOcide PH Phenoxyethanol, Hydroxyacetophenone, Caprylyl | | | | 1.0 | | | | | | | |
| Glycol | | | | | | | | | | | |
| SymOcide PS Phenoxyethanol, Decylene Glycol, 1,2-Hexanediol | | | 2.0 | | | | | | | | |
| SymOleo ® Vita7 Glycine soja oil, Gossypium herbaceum seed oil, | | | | 1.0 | | | | | | | |
| mangifera idica seed butter, olea europaea fruitoil, persea gratissima | | | | | | | | | | | |
| oil, prununs amygdalus dulcis (sweet almond) oil, Theobroma cacao | | | | | | | | | | | |
| seed butter | | | | | | | | | | | |
| SymPeptide 225 Glycerin, Aqua, Myristoyl Pentapeptide-11 | | 1.0 | | | | | | | | | |
| SymRadiance 377 Phenylethyl Resorcinol | 0.1 | | | | | | | | | | |
| SymReboot L19 Maltodextrin, Lactobacillus Ferment | | | | | | 0.2 | 0.3 | | | | |
| SymRelief 100 Bisabolol, Zingiber officinale root extract | | | | | | 0.1 | | | | | |
| SymRenew ™ HPR Diisopropyl Adipate, Hydroxypinacolone | | | | | | | 1.0 | | | | |
| Retinoate, Tocopherol | | | | | | | | | | | |
| SymRepair 100 Hexyldecanol, Bisabolo, Cetylhydroxyproline | | | | | | | | | 1.0 | | |
| Palmitade, Stearic Acid, Brassica Campestris (Rapeseed) Sterols | | | | | | | | | | | |
| SymSave H Hydroxyacetophenone | | 0.6 | | | 0.5 | 0.5 | | 0.5 | | | |
| SymSitive 1609 Pentylene Glycol, 4-t-Butylcyclohexanol | | | | | | | | | 1.0 | | |
| SymSoft Scalp Trideceth-9 (and) PEG-5 Ethylhexanoate (and) | | | | | 1.0 | | | | | | |
| Water (and) Hydroxyphenyl Propamidobenzoic Acid | | | | | | | | | | | |
| SymSol PF-3 Aqua, Pentylene Glycol, Sodium Lauryl Sulfoacetate, | | | | | | | | 1.5 | | | |
| Sodium Oleoyl Sarcosinte, Sodium Chloride, Sodium Oleate | | | | | | | | | | | |
| SymUrban Benzylidene Dimethoxydimetylindanone | | 0.3 | | | | | | | | | |
| SymVital AR 3040 Zinbiber officinale root extract | | 0.2 | | | | | | | | | |
| Tagat L2 PEG-20 Glyceryl Laurate | | | | | | | | | | 2.5 | |
| Tapioca Pure Tapioca Starch | | 2.0 | | | | | | | | | |
| Tegin ® BL 315 Glycol Distearate | | | | 1.5 | | | | | | | |
| Tegosoft TN C12-15 alkyl benzoate | | 11.0 | | | | | | | | | |
| Texapon N70 Sodium Laureth Sulfate | | | | 16.0 | 35.0 | | | | | | |
| Tocopheryl Acetate Tocopheryl Acetate | | | | | | 0.15 | | | | | 0.3 |
| Uvasorb HEB Diethylhexyl butamido triazone | | 2.0 | | | | | | | | | |
| Vegetable oil triglyceride Caprylic/Capric Triglyceride | 6.0 | | | | | | | | | | |
| Water Aqua | Ad | Ad | Ad | Ad | Ad | Ad | | Ad | Ad | Ad | Ad |
| | 100 | 100 | 100 | 100 | 100 | 100 | | 100 | 100 | 100 | 100 |
| Xanthan Gum Xanthan Gum | 0.4 | 0.2 | | | | 0.5 | 0.2 | 0.1 | 0.1 | | |
| Xiameter ® PMX-0345 Cyclosiloxane Cyclopentasiloxane, | | | | | | | | | 0.5 | | |
| Cyclohexasiloxane | | | | | | | | | | | |
| Zinc Sulfate Heptahydrate Zinc Sulfate | | | | | | | | | | | 1.0 |

TABLE 20
Cosmetic formulations 12 to 17 (amounts in % b.w.)

| | Amounts [wt.-%] | | | | | |
|---|---|---|---|---|---|---|
| Ingredients INCI name | 12 | 13 | 14 | 15 | 16 | 17 |
| 3,4-Dicaffeoylquinic acid | 0.001 | | | | | |
| 3,5-Dicaffeoylquinic acid | 0.001 | | | | | |
| 4,5-Dicaffeoylquinic acid | | | | | | |
| Yarrow Extract Powder (40% extract dry matter) Maltodextrin, Achillea Millefolium Extract | | 0.15 | | 0.15 | | 0.2 |
| Arnica flower extract powder (60% extract dry matter) Arnica montana extract, Maltodextrin | | | 0.3 | | | |
| Elderflower Extract Powder Sambus nigra Extract | 0.3 | | | | 0.1 | |
| Ivy Leaf Extract Hedera helix Extract | | 0.1 | | | | |
| Tocopherol Tocopherol | 0.3 | 0.1 | 0.5 | 0.1 | 0.1 | 0.3 |
| Aloe Vera Gel Concentrate 10/1 Aloe Barbadensis Leaf Juice | | 0.3 | | | | |
| Abil 350 Dimethicone | | | | 2.0 | | |
| Allantoin Allantoin | | | | 0.2 | | |
| AMISOFT ® CS-11/CS-11(F) SODIUM COCOYL GLUTAMATE | | | | | 0.5 | |
| Anisic Acid Anisic Acid | | | | 0.4 | | |
| Carbopol ® Aqua SF-1 Polymer ACRYLATES COPOLYMER | | | | | 10.0 | |
| Carbopol Ultrez-10 Carbomer | | | | 0.1 | | |
| Ceramide Bio Cetylhydroxyproline Palmitamide | | | | 0.1 | | |
| Citric acid 10% solution Aqua, Citric acid | 1.7 | | | 0.4 | | |
| Coconut oil refined Cocos Nucifera (Coconut) Oil | 10.0 | | | | | |
| Colour | | 0.04 | | | | |
| Covathick 2009 Microcrystalline Cellulose, Cellulose Gum | 0.5 | | | | | |
| Cutina GMS V Glyceryl Stearate | 1.0 | | | 2.0 | | |
| Dermofeel BGC Butylene Glycol Dicaprylate/Dicarate | 5.0 | | | | | |
| Dermofeel sensolv MB/soamyl Laurate | | | 4.5 | | | |
| Dermosoft 1388 eco Glycerin, Aqua, Sodium Levulinate, Sodium Anisate | 3.0 | | | | | |
| Dracorin CE Glyceryl Stearate Citrate | | 5.0 | | | | |
| Drago-Oat-Active Water (Aqua), Butylene Glycol, Avena Sativa (Oat) Kernel Extract | 2.0 | | | | | |
| Dragoxat 89 Ethylhexyl Isononanoate | | | | | | 2.0 |
| Dragosantol Bisabolol | 0.15 | | | | | |
| Dragosine Carnosine | | | | | | 0.2 |
| EDTA BD Disodium EDTA | | 0.1 | | | 0.1 | 0.1 |
| Emulsiphos Potassium Cetyl Phosphate, Hydrogenated Palm Glycerides | | | | 2.0 | | 2.5 |
| Ethanol Ethanol | 5.0 | | | | | |
| Extrapone Green Tea GW Glycerin, Water (Aqua), Camellia Sinensis Leaf Extract | | | | 0.2 | | |
| Extrapone Arnica GW Glycerin, Water (Aqua), Arnica Montana Flower Extract | | | | 1.0 | | |
| Farmal MS6135 Calcium Starch Octenylsuccinate | | | | | | 2.0 |
| Floraesters K-100 ® Jojoba Hydrolyzed jojoba esters, Jojoba esters, Aqua | | | | | | 0.5 |
| Fragrance Parfum | 0.3 | 0.5 | | 0.2 | | 0.5 |
| Frescolat ML Menthyl Lactate | | | | | 0.3 | |
| Frescolat Plus Menthol, Menthyl Lactate | | 0.2 | | | | |
| Frescolat X-Cool Menthyl Ethylamido Oxalate | 1.0 | | | | | |
| Genuvisco CG-131 Carrageenan | 0.3 | | | | | |
| Glycerin Glycerin | 3.0 | 3.0 | | 2.0 | | |
| Hydrolite 5 green 1,2-Pentanediol | | | 3.0 | | 1.0 | |
| Hydrolite 6 1,2-Hexanediol | | | | | 0.5 | |
| Hydrolite 7 green 1,2-Heptanediol | 0.5 | | | | | 0.5 |
| Hydrolite CG 1,2-Octanediol | | | 0.5 | | | |
| Hydroviton 24 Water, Glycerin, Sodium Lactate, TEA Lactate, Serine, Lactic Acid, Urea, Sorbitol, | | 1.0 | | | | |
| Sodium Chloride, Lauryl Diethylenedi-aminoglycine, Lauryl Aminopropyl-glycine, Allantoin | | | | | | |
| Isodragol Triisononanoin | | | | 2.0 | | |
| Isolan GPS Polyglyceryl-4 Diisostearate/Polyhydroxystearate/Sebacate | | | 3.5 | | | |
| Kelogel F Gellan Gum | | 0.05 | | | | |
| Lanette 16 Cetyl Alcohol | | 5.0 | | | | |
| Lanette O Cetearyl Alcohol | | | | 3.0 | | |
| Passion Fruit Oil Refined Passiflora Edulis Seed Oil | | 2.0 | | | | |
| Neo-Heliopan 303 Octocrylene | | | | | | 10.0 |
| Neo-Heliopan 357 Butyl Methoxydibenzoylmethane | | | | | | 4.5 |
| Neo-Heliopan BMT Bis-Ethylhexyloxyphenol Methoxypenyl Triazine | | | 3.0 | | | 2.5 |
| Neo-Heliopan HMS Homosalate | | | 5.0 | | | 10.0 |
| Neo-Heliopan OS Ethylhexyl Salicylate | | | 4.0 | | | 5.0 |
| Neo PCL wssl, N Trideceth-9, PEG-5 Ethylhexanoate, Water | | | | 1.0 | | |
| PCL Liquid 100 Cetearyl Ethylhexanoate | | | | 5.0 | | |
| PCL Solid Stearyl Heptanoate, Stearyl Caprylate | | | | 2.0 | | |
| Plant oil triglyceride Caprylic/Capric Triglyceride | | 3.0 | | | | |
| Prisorine 3505 Isostearic acid | | | | | | 1.5 |
| Propylene Glycol Propylene Glycol | | | | 5.0 | | |
| Sodium Hydroxide 10% solution Aqua, Sodium Hydroxide | | 0.15 | | 0.3 | 2.0 | |
| Softisan 100 Hydrogenated Coco-Glycerides | | 6.0 | | | | |
| SymCalmin Butylene Glycol, Pentylene Glycol, Hydroxyphenyl Propamidobenzoic Acid | | | | 2.0 | | |
| SymDiol 68 1,2-Hexanediol, Caprylyl glycol | | 0.5 | | 1.0 | | |
| SymDecanox HA Caprylic/Capric Triglyceride, Hydroxymethoxyphenyl Decanone | | | | | | 1.0 |
| SymDStress Cannabidiol | 0.05 | | | | | |
| SymMatrix Maltodextrin, Rubus Fruticosus Leaf Extract | | | | 0.1 | | |
| SymMollient PDCC Propanediol Dicaprylate/Caprate | | | | | | 3.5 |
| SymMollient S Cetearyl Nonanoate | | 1.0 | | | | |
| SymOcide C o-cymen-5-o/ | | | | 0.1 | | |
| SymOcide PH Phenoxyethanol, Hydroxyacetophenone, Caprylyl Glycol, Aqua | | | | | | 1.4 |
| SymRelief S Bisabolol, Hydroxymethoxyphenyl Decanone | | | | 0.2 | | |
| SymSave H Hydroxyacetophenone | | 0.5 | | | 0.5 | |
| SymSitive 1609 Pentylene Glycol, 4-t-Butylcyclohexanol | | | | 1.5 | | |
| SymSol PF-3 Water (Aqua), Sodium Lauryl Sulfoacetate, Pentylene Glycol, Sodium Oleoyl | | | | | 3.0 | 2.0 |
| Sarcosinate, Sodium Chloride, Sodium Oleate | | | | | | |
| SymUrban Benzylidene Dimethoxydimetylindanone | | | | | | 0.2 |
| SymVital AR 3040 Zinbiber officinale root extract | | | | | 0.1 | 0.1 |
| Tapioca Pure Tapioca Starch | | | | | | 1.5 |
| Tego Care CG90 Cetearyl Glucoside | 1.0 | | | | | |
| TEGOSOFT DC MB Decyl Cocoate | | | 4.0 | | | |
| Tocopheryl Acetate Tocopheryl Acetate | | 0.15 | | | | |
| Uvinul A plus Diethylamino Hydroxybenzoyl Hexyl Benzoate | | | 3.0 | | | |
| Uvinul T150 Ethylhexyl Triazone | | | 3.0 | | | |
| Verstatil TBO Triethylcitrate; Caprylyl Glycol; benzoic Acid | | | 1.0 | | | |
| Vivapur CS Tex sun Microcrystalline Cellulose, Cellulose Gum | | | | | | 1.0 |
| Vivapur CS 032 XV Microcrystalline Cellulose, Xanthan Gum | | | | | | 1.0 |
| Water Aqua | Ad | Ad | Ad | Ad | Ad | Ad |
| | 100 | 100 | 100 | 100 | 100 | 100 |
| Xanthan Gum Xanthan Gum | 0.3 | | | 0.1 | | 0.3 |
| Yerba Mate Leaf in Glycerin Glycerin, Water, llex Paraguariensis Leaf Extract | | 2.0 | | | | |
| Zinc Stearate Zinc Stearate | | | 0.5 | | | |
| Zinc Sulfate Heptahydrate Zinc Sulfate | | | 1.2 | | | |

Formulation 18: Gel dental cream

| Ingredients | I (%) | II (%) | III (%) |
|---|---|---|---|
| Sodium carboxymethylcellulose | 0.40 | 0.40 | 0.40 |
| Sorbitol 70% in water | 72.00 | 72.00 | 72.00 |
| Polyethylenglycol (PEG) 1500 | 3.00 | 3.00 | 3.00 |
| Sodium saccharinate | 0.07 | 0.07 | 0.07 |
| Sodium fluoride | 0.24 | 0.24 | 0.24 |
| p-Hydroxybenzoic acid (PHB) | 0.15 | 0.15 | |
| ethylester | | | |
| SymDiol 68 | | | 0.5 |
| SymSave H | | | 0.25 |
| Peppermint flavour | 1.00 | 1.00 | 1.00 |
| Yarrow (Achillea millefolium) | 0.40 | | 0.10 |
| extract powder (25% yarrow | | | |
| juice dry matter, 75% | | | |
| maltodextrin) | | | |
| 3,4-dicaffeoylquinic acid | | 0.002 | |
| Tocopherol | 0.20 | 0.05 | 0.10 |
| Abrasive Silica | 11.00 | 11.00 | 11.00 |
| Thickening Silica | 6.00 | 6.00 | 6.00 |
| Sodium dodecylsulfate (SDS) | 1.40 | 1.40 | 1.40 |
| Distilled water | ad 100.00 | ad 100.00 | ad 100.00 |

Formulation 19: Ready-to-use mouthwash with fluoride

| Ingredients | I (%) | II (%) | III (%) |
|---|---|---|---|
| Ethanol | 7.00 | 7.00 | |
| Glycerin | 12.00 | 12.00 | |
| Sodium fluoride | 0.05 | 0.05 | 0.18 |
| Pluronic F-127 ® (BASF, | 1.40 | 1.40 | |
| surface active substance) | | | |
| Sodium phosphate buffer pH 7.0 | 1.10 | 1.10 | |
| Sorbic acid | 0.20 | 0.20 | |
| Sodium saccharinate | 0.10 | 0.10 | 0.10 |
| Cinnamon/menthol flavour | 0.15 | 0.15 | 0.15 |
| Elderflower (Sambus nigra) extract | 0.25 | | 0.10 |
| powder standardized to ≥0.5% | | | |
| dicaffeoylquinic in sum Arnica | | | |
| (Arnica montana) flower extract | | | 0.10 |
| 4,5-dicaffeoylquinic acid | | 0.001 | |
| Tocopherol | 0.40 | 0.05 | 0.20 |
| Colour | 0.01 | 0.01 | 0.01 |
| Sorbitol 70% | | | 10 |
| Cremophor RH455 | | | 1.8 |
| SymDiol 68 | | | 0.5 |
| SymSave H | | | 0.2 |
| Distilled water | ad 100.00 | ad 100.00 | ad 100.00 |

Formulation 20: Chewing gums

| | I (%) | II (%) | III (%) |
|---|---|---|---|
| Chewing gum base | 21.00 | 21.00 | 21.00 |
| Glucose syrup | 16.50 | 16.50 | 16.50 |
| Glycerin | 0.50 | 0.50 | 0.50 |
| Powdered sugar | 60.45 | 60.36 | 60.27 |
| Spearmint aroma | 1.50 | 1.50 | 1.50 |
| Elderflower (Sambus nigra) extract | 0.15 | 0.30 | |
| Yarrow (Achillea millefolium) extract powder | 0.05 | | |
| 3,5-Dicaffeoylquinic acid | | | 0.002 |
| Tocopherol | 0.25 | 0.05 | 0.10 |

1-15. (canceled)

16. A mixture comprising:
(a) one or more dicaffeoylquinic acids; and
(b) tocopherol;
wherein (a) and (b) are in a weight ratio of 1:1000 to 5:1.

17. The mixture of claim 16, wherein the one or more dicaffeoylquinic acids are selected from 3,4-dicaffeoylquinic acid, 3,5-dicaffeoylquinic acid, 1,5-dicaffeoylquinic acid, 4,5-dicaffeoylquinic acid, or mixtures thereof.

18. The mixture of claim 16, wherein the mixture comprises 0.1 to 85 wt. % of the one or more dicaffeoylquinic acids, based on a total weight of the mixture.

19. The mixture of claim 16, wherein the mixture comprises 15 to 99.9 wt. % of the tocopherol, based on the total weight of the mixture.

20. A composition comprising the mixture of claim 16.

21. The composition of claim 20, wherein (a) and (b) are in a weight ratio of 1:1000 to 5:1.

22. The composition of claim 20, wherein the composition comprises one or more plant extracts.

23. The composition of claim 20, wherein the composition comprises 0.000001 to 75 wt. % of the one or more dicaffeoylquinic acids and/or 0.001 to 99 wt. % of the tocopherol, based on a total weight of the composition.

24. The composition of claim 20, wherein the composition is a pharmaceutical composition appropriate for use as a medicament.

25. A method for stimulating expression of one or more heat shock proteins, reducing or preventing skin aging, increasing thermotolerance of skin, increasing resistance of skin to low humidity, improving barrier function of keratinocytes, increasing skin moisture, or a combination thereof, the method comprising administering one or more dicaffeoylquinic acids to the skin and stimulating the expression of the one or more heat shock proteins, reducing or preventing the skin aging, increasing the thermotolerance of the skin, increasing the resistance of the skin to low humidity, improving the barrier function of the keratinocytes, increasing the skin moisture, or a combination thereof.

26. The method of claim 25, wherein the one or more dicaffeoylquinic acids are selected from 3,4-dicaffeoylquinic acid, 3,5-dicaffeoylquinic acid, 1,5-dicaffeoylquinic acid, 4,5-dicaffeoylquinic acid, or mixtures thereof.

27. A method for stimulating expression of one or more heat shock proteins, reducing or preventing skin aging, increasing thermotolerance of skin, increasing resistance of skin to low humidity, improving barrier function of keratinocytes, increasing skin moisture, or a combination thereof, the method comprising administering the mixture of claim 16 to the skin and stimulating the expression of the one or more heat shock proteins, reducing or preventing the skin aging, increasing the thermotolerance of the skin, increasing the resistance of the skin to low humidity, improving the barrier function of the keratinocytes, increasing the skin moisture, or a combination thereof.

28. The method of claim 27, wherein the one or more dicaffeoylquinic acids are selected from 3,4-dicaffeoylquinic acid, 3,5-dicaffeoylquinic acid, 1,5-dicaffeoylquinic acid, 4,5-dicaffeoylquinic acid, or mixtures thereof.

29. The method of claim 27, wherein the method induces the expression of one or more genes selected from HSPB2, HSPB8, CRYAB, CLDN1, OCLN, DEFB1, DSC1, DSG1, or combinations thereof.

30. The method of claim 27, wherein the method synergistically stimulates the expression of the one or more heat shock proteins, reduces or prevents the skin aging, increases the thermotolerance of the skin, increases the resistance of the skin to low humidity, improves the barrier function of the keratinocytes, increases the skin moisture, or a combination thereof.

31. A method for stimulating expression of one or more heat shock proteins, reducing or preventing skin aging, increasing thermotolerance of skin, increasing resistance of skin to low humidity, improving barrier function of keratinocytes, increasing skin moisture, or a combination thereof, the method comprising administering the composition of claim 20 to the skin and stimulating the expression of the one or more heat shock proteins, reducing or preventing the skin aging, increasing the thermotolerance of the skin, increasing the resistance of the skin to low humidity, improving the barrier function of the keratinocytes, increasing the skin moisture, or a combination thereof.

32. The method of claim 31, wherein the one or more dicaffeoylquinic acids are selected from 3,4-dicaffeoylquinic acid, 3,5-dicaffeoylquinic acid, 1,5-dicaffeoylquinic acid, 4,5-dicaffeoylquinic acid, or mixtures thereof.

33. The method of claim 31, wherein the method induces the expression of one or more genes selected from HSPB2, HSPB8, CRYAB, CLDN1, OCLN, DEFB1, DSC1, DSG1, or combinations thereof.

34. The method of claim 31, wherein the method synergistically stimulates the expression of the one or more heat shock proteins, reduces or prevents the skin aging, increases the thermotolerance of the skin, increases the resistance of the skin to low humidity, improves the barrier function of the keratinocytes, increases the skin moisture, or a combination thereof.

35. A method for increasing the stability of one or more dicaffeoylquinic acids comprising combining the one or more dicaffeoylquinic acids with tocopherol.
